(19) United States
(12) Patent Application Publication
(10) Pub. No.: US 2017/0303555 A1
(43) Pub. Date: Oct. 26, 2017

(54) SYSTEM AND METHOD FOR RADIATION TREATMENT WITH SPINNER

(71) Applicant: AseptoRay Ltd., Ma'a lot Tarshiha (IL)

(72) Inventors: Dror LIVNE, Kibbutz Cabri (IL); Amir TRIFMAN, Kiryat Tivon (IL); Mordechai KOREN, Givat Ada (IL)

(73) Assignee: AseptoRay Ltd., Ma'a lot Tarshiha (IL)

(21) Appl. No.: 15/646,642

(22) Filed: Jul. 11, 2017

Related U.S. Application Data
(63) Continuation-in-part of application No. PCT/IL2015/050037, filed on Jan. 11, 2015.

(51) Int. Cl. A23C 3/07; B01J 19/12; A23L 3/26; C02F 1/32; A61L 2/08

(52) U.S. Cl. CPC A23C 3/076; B01J 19/12; A23L 3/26; A61L 2/08; C02F 1/325

(57) ABSTRACT

A radiation treatment unit is provided. The radiation treatment unit includes a pipe sub-system that includes a helically-coiled pipe for passing therethrough a fluid to be treated and defining therein a cylindrical cavity. The radiation treatment unit also includes at least one radiation sub-system configured to generate radiation and disposed so as to expose the pipe sub-system to the radiation. The pipe is provided with a spinner that includes smoothly twisting spinning surfaces spanning between ends thereof.

### CROSS-REFERENCE TO RELATED APPLICATION

This application is a continuation-in-part of International Application No. PCT/IL2015/050037, filed Jan. 11, 2015, the full contents of which are incorporated herein by reference.

### TECHNOLOGICAL FIELD

The presently disclosed subject matter relates to a radiation treatment system, and in particular Dean flow reactor.

### BACKGROUND

Ultraviolet light (UV-C) treatment is one of the nonthermal technologies to kill microorganisms. Photochemical changes in proteins and nucleic acids are responsible for inactivation of microorganisms when UV light is absorbed by the food during the UV treatment process.

Thus, ultraviolet light (UV) holds considerable promise in food processing as an alternative to traditional thermal processing. Its applications include pasteurization of liquid food, such as milk and juices, post lethality treatment for meats, treatment of food contact surfaces and to extend the shelf-life of fresh produce.

The disinfection of high UV transmissible fluids such as water is relatively easy given the high penetration of photons into the matrix. However, with opaque fluids such as milk the penetration of UV photons is limited to the subsurface. Consequently, to facilitate the UV treatment of milk it is necessary to ensure efficient mixing so that each part of the fluid is exposed to UV.

It is generally known to perform UV treatment of milk in reactors including coiled tubes since these provide secondary vortices and can cause superior mixing of fluid flowing through the reactor. One such reactor is disclosed for example in Tucker (U.S. Pat. No. 4,798,702), and it comprises a coil of corrugated pipe wrapped around a germicidal radiation source into the shape of a helix.

### General Description

Key factors influencing efficiency of UV-C treatment include reactor design, fluid dynamic parameters and UV-C absorbance of liquid food. Turbulent flow of liquid foods in continuous flow UV reactors increases inactivation of microorganisms in fresh juices, wine, liquid egg whites, beer, and milk, process water specialty turbid and unclear water. Reynolds number (Re) is a measure of the ratio of inertial forces to viscous forces for fluid flow in a coiled tube. It is expressed as:

Re=(ρ/μ)×vD (1)

where Re is the Reynolds number, ρ is density of fluid, μ is dynamic viscosity of fluid, D is diameter of tube carrying the fluid, and v is velocity of flow. Laminar flow occurs when Re<2100, whereas Re>4000 indicates turbulent flow. Flow with Re between these numbers is considered transient flow.

The Dean number De is a similarity parameter governing the fluid motion in coiled tube flow configuration:

De=Re\sqrt{D/Dc} (2)

where D is the tube diameter, Dc is the coil diameter, and Re is the tube Reynolds number. When the flow of a fluid in a coiled tube reactor is accompanied by secondary flow vortices, it is called Dean flow. This occurs when the ratio (D/Dc) in Equation (2) is between 0.03 and 0.1 (Dean, 1927).

As mentioned above, another key factor in fluencing efficiency of UV-C treatment is UV-C absorbance of liquid food, i.e. the effectiveness of UV irradiation, defined by applied intensity and contact time, satisfying:

F=i×t (3)

where i is the intensity of the applied UV light source(s) and t is the time of the exposure of the fluid to the above UV light.

In accordance with one aspect of the presently disclosed subject matter there is provided a radiation treatment unit having an elongated shape and a central longitudinal axis comprising:
(a) a first pipe assembly comprising:
   a first coiled pipe for passing therethrough a fluid to be treated and having a first coil inner cylindrical surface defining a first cylindrical cavity;
   a first radiation source disposed within the first cylindrical cavity; and
   a first support assembly supporting at least the first coiled pipe,
(b) a second pipe assembly comprising:
   a second coiled pipe for passing therethrough a fluid to be treated and having a second coil inner cylindrical surface defining a second cylindrical cavity,
   a second radiation source disposed within the second cylindrical cavity; and
   a second support assembly supporting at least the second coiled pipe;
the second pipe assembly being configured to be coaxially inserted within the first cylindrical cavity so that the second coiled pipe is disposed between the first radiation source and the second radiation source, so that a fluid passing through the first coiled pipe is exposed to an inside radiation of the first radiation source and a fluid passing through the second coiled pipe is exposed to an outside radiation from the first radiation source and an inside radiation from the second radiation source;
one or both of the pipe assemblies being provided with a spinner therein, the spinner comprising smoothly twisting spinning surfaces spanning between ends thereof.

The axial length of the spinner may be much less than the length of the pipe (i.e., the length of the fluid path defined thereby). For example, it may be no greater than about 1% of the length of the pipe. According to other examples, it may be no greater than about 1/150^{th} the length of the pipe.

The spinning surfaces may be twisted through substantially 360° between the ends of the spinner.

The spinning surfaces may span between two helical edges.

The helical edges may tightly fit within the inner cylindrical surface of its respective pipe assembly.

The pitch of the spinning surfaces may be substantially constant between the ends of the spinner.

The pitch of the spinning surfaces may decrease along its length.

The spinner may be made of stainless steel.

The parameters of the radiation treatment unit can be such that a radiation doze applied to a first treated fluid during its passage through the first coiled pipe is substantially equal to a radiation doze applied to a second treated fluid during its passage through the second coiled pipe.

The parameters are selected from the group can consist of: intensity of the first radiation source, intensity of the second radiation source, time of exposure to the first radiation source of a fluid passing through the first coiled pipe, time of exposure to the second radiation source of a fluid passing through the second coiled pipe, inner and/or outer diameter of the first coiled pipe, inner and/or outer diameter of the second coiled pipe, outer diameter of a first coil defined by the first coiled pipe, outer diameter of a second coil defined by the second coiled pipe, a flow rate of a fluid passing through the first coiled pipe and a flow rate of a fluid passing through the second coiled pipe.

The radiation treatment unit can further comprise a unit holding assembly configured for supporting the first pipe assembly and the second pipe assembly.

The unit holding assembly can be configured for allowing an axial movement of at least one of the first pipe assembly and the second pipe assembly with respect to the other of the first pipe assembly and the second pipe assembly.

The unit holding assembly can comprise a sliding mechanism allowing the first pipe assembly to slide along the longitudinal axis with respect to the second pipe assembly, allowing thereby an access to the second pipe assembly.

The first support assembly can comprise a couple of first end supports disposed adjacent ends of the radiation treatment unit and a plurality of first rods extending between the first end supports and configured for holding the first coiled pipe.

The first radiation source can be configured to be fixed to the first end supports.

The second support assembly can comprise a couple of second end supports disposed adjacent ends of the radiation treatment unit and a plurality of second rods extending between the second end supports and configured for holding the second coiled pipe.

The second radiation source can be configured to be fixed to the second end supports.

The first and the second radiation sources can be equally spaced from the second coiled pipe.

A single support assembly can serve as both the first support assembly and the second support assembly.

The first and the second coiled pipes can be equally spaced from the first radiation source.

The first coiled pipe can comprise a first smooth inner surface of a diameter D_{I} and a first coil outer cylindrical surface of a diameter D_{c1}, satisfying the condition 0.03≦D_{I}/D_{c1}≦0.1.

The first coiled pipe can satisfy the condition D_{I}/D_{c1}=0.0.3.

The second coiled pipe can comprise a second smooth inner surface of a diameter D₂ and a second coil outer cylindrical surface of a diameter D_{c2}, satisfying the condition 0.03≦D₂/D_{c2}≦0.1.

The second coiled pipe can satisfy the condition D₂/D_{c2}=0.0.5.

The first and the second radiation sources can comprise a plurality of UV lamps.

Each of the lamps can be mounted separately to at least one of the first support assembly and the second support assembly.

The radiation treatment unit can further comprise a separating element configured for receiving electrical cables of the first and the second radiation sources.

In accordance with another aspect of the presently disclosed subject matter there is provided a radiation treatment unit having an elongated shape and a central longitudinal axis, comprising:
(a) a first pipe assembly comprising:
   a first coiled pipe for passing therethrough a fluid to be treated and having a first coil inner cylindrical surface defining a first cylindrical cavity;
   a first radiation source disposed within the first cylindrical cavity; and
   a first support assembly supporting the first coiled pipe and the first radiation source,
(b) a second pipe assembly comprising:
   a second coiled pipe for passing therethrough a fluid to be treated and having a second coil inner cylindrical surface defining a second cylindrical cavity,
   a second radiation source disposed within the second cylindrical cavity; and
   a second support assembly supporting at the second coiled pipe and the second radiation source;
the second pipe assembly being configured to be coaxially inserted within the first cylindrical cavity so that the second coiled pipe is disposed between the first radiation source and the second radiation source, so that a fluid passing through the first coiled pipe is exposed to an inside radiation of the first radiation source and a fluid passing through the second coiled pipe is exposed to an outside radiation from the first radiation source and an inside radiation from the second radiation source;
a unit holding assembly configured for supporting the first pipe assembly and the second pipe assembly, to allow an axial movement of at least one of the first pipe assembly and the second pipe assembly with respect to the other of the first pipe assembly and the second pipe assembly;
one or both of the pipe assemblies being provided with a spinner therein, the spinner comprising smoothly twisting spinning surfaces spanning between ends thereof. The spinner may be as described above, mutatis mutandis.

In accordance with another aspect of the presently disclosed subject matter there is provided a radiation treatment unit having an elongated shape, the unit comprising:
(a) a pipe sub-system comprising a pipe for passing therethrough a fluid to be treated, the pipe having an inner diameter d, a thickness t of its wall, an outer surface and a smooth inner surface of a diameter D and extending between two opposite ends spaced from each other by a distance L in a helically coiled manner so that the coiled pipe has a coil inner cylindrical surface defining a cylindrical cavity and a coil outer cylindrical surface of a diameter D_{c};
(b) an inner radiation sub-system disposed within the cylindrical cavity and an outer radiation sub-system disposed outside the coil outer cylindrical surface, each comprising at least one inner radiation source extending along a length corresponding to the length L;
   wherein the parameters have the following values:
L=1,200-1,700 mm
d=10 to 25 mm;
t=0.63-1.27 mm
D=10-25 mm; and
D_{c} meeting the condition: 0.03≦D/D_{c}≦0.1;
   the pipe being provided with a spinner therein, the spinner comprising smoothly twisting spinning surfaces spanning between ends thereof. The spinner may be as described above, mutatis mutandis.

The pipe may satisfy the condition 0.03≦D/D_{c}≦0.1, and more particularly D/D_{c}=0.05 or D/D_{c}=0.04.

The pipe's inner diameter can be in the range 10.5-12.5 mm, more particularly, in the range of 10.8-11.5 mm, more particularly, in the range of 11-11.2 for 1000 L/H system.

The pipe's inner diameter can be in the range 15.5-19.5 mm, more particularly, in the range of 16.5-17.5 mm, more particularly, in the range of 17-17.2 for 3000 L/H system.

The pipe's inner diameter can be in the range 20.5-23.5 mm, more particularly, in the range of 20.8-21.5 mm, more particularly, in the range of 21-21.2 for 5000 L/H system.

The wall thickness of the pipe can be in the range 0.7-1 mm, more particularly, in the range of 0.8-0.82 mm.

The pipe can be made of Fluoropolymers, and more particularly, from Fluorinated Ethylene Propylene (FEP).

The inner and the outer radiation sources can be spaced from the coil.

The spacing between the coil and the corresponding radiation source can be between 0.5 and 4 cm, more particularly, in the range of 0.8-3 cm, and more particularly, in the range of 1 to 2 cm.

The inner and the outer radiation sources can be equally spaced from the coil.

The inner and the outer radiation sources can comprise a plurality of UV lamps.

Each of the lamps can be mounted separately at the ends of the unit.

At least some of the lamps can have a length in the range of 200-1,700 mm, more specifically in the range of 800-1,600 mm, and more specifically in the range of 1,500-1,600 mm.

At least some of the lamps can have an intensity in the range of 500-1,300 μW/cm², according to some examples in the range of 720 to 780 μW/cm², and according to other examples in the range of 1,100 to 1,200 μW/cm².

The radiation treatment unit can have a central axis extending between two opposite ends of the unit, wherein the sub-systems of the radiation treatment unit are coaxially disposed with respect to the central axis.

The radiation treatment unit can further comprise a unit holding assembly configured for fixedly mounting thereon the sub-systems of the unit.

The unit holding assembly can comprise two end supports and a plurality of outer fixation rods fixedly connected thereto and extending along a longitudinal axis of the system, at a radial distance therefrom greater than that of the radiation sources and the coiled pipe.

The unit holding assembly further can comprise a central fixation rod extending between the end supports and coaxial with the longitudinal axis.

The end supports can receive therein ends of the radiation sources of both the inner and the outer radiation sub-systems.

The unit holding assembly further can comprise an intermediate support disposed between the end supports, the radiation sources passing through corresponding openings in the intermediate support.

The intermediate support can comprise an intermediate lateral support and an intermediate central support, wherein the at least one radiation source of the inner radiation sub-system passes through corresponding to at least one opening in the central support, the at least one outer radiation source passes through corresponding to at least one opening in the intermediate outer support.

The outer rods can pass through corresponding openings in the lateral support and the central rod passes through a corresponding opening in the intermediate support.

The unit holding assembly can further comprise coil pipe supporting brackets projecting radially from the intermediate central support, the brackets having ends optionally received in the end supports.

The coiled pipe can pass through an area between the central and the lateral intermediate supports so that its inner cylindrical surface is in contact with the supporting brackets and its outer cylindrical surface is in contact with the intermediate lateral support.

In accordance with another aspect of the presently disclosed subject matter there is provided a radiation treatment unit having an elongated shape, the unit comprising:
(a) a pipe sub-system comprising a pipe for passing therethrough a fluid to be treated, the fluid having a density p and a velocity V, the pipe having an outer surface and a smooth inner surface of a diameter D and extending between two opposite ends spaced from each other by a distance L in a helically coiled manner so that the coiled pipe has a coil inner cylindrical surface defining a cylindrical cavity and a coil outer cylindrical surface of a diameter D_{c};
(b) an inner radiation sub-system disposed within the cylindrical cavity and an outer radiation sub-system disposed outside the coil outer cylindrical surface, each comprising at least one inner radiation source extending along a length corresponding to the length L;
   wherein the coiled pipe is configured to withstand pressure difference ΔP which does not extend 12 bar in by means of its parameters meeting at least the following conditions:
0.03≦D/D_{c}≦0.1;
ΔP=fρV²/200000·L/D
   wherein the parameter f is a friction factor;

the pipe being provided with a spinner therein, the spinner comprising smoothly twisting spinning surfaces spanning between ends thereof. The spinner may be as described above, mutatis mutandis.

The parameter f can be calculated in accordance with the following equation:

$$f = {[ \frac{1}{- 2 log ( \frac{\frac{ε}{D}}{3.7} ) + ( \frac{2.51}{Re \sqrt{f}} )} ]}^{2} \qquad ( 4 )$$

wherein e is absolute roughness of the pipe.

The above calculation of the friction factor f is suitable for coiled pipes, such as the pipe of the presently disclosed subject matter.

In accordance with another aspect of the presently disclosed subject matter there is provided a method for using one or more of the above radiation treatment units, comprising passing through the pipe the fluid to be treated thereby exposing it to the radiation for 20 to 30 seconds.

The exposure time can be in the range of 24-26 seconds, and more specifically 25 seconds.

In accordance with another aspect of the presently disclosed subject matter, there is provided a radiation treatment unit comprising a pipe for passing therethrough a fluid to be treated and disposed so as to be exposed to a radiation source, the pipe comprising a spinner therein, the spinner comprising smoothly twisting spinning surfaces spanning between ends thereof.

The spinning surfaces may be twisted through substantially 360° between the ends of the spinner.

The spinning surfaces may span between two helical edges.

The helical edges may tightly fit within the inner cylindrical surface of its respective pipe assembly.

The pitch of the spinning surfaces may be substantially constant between the ends of the spinner.

The pitch of the spinning surfaces may decrease along its length.

The spinner may be made of stainless steel.

The pipe may be constructed so as to satisfy the Dean ratio.

The radiation source may comprise an ultraviolet light source.

In accordance with another aspect of the presently disclosed subject matter there is provided a radiation treatment system comprising at least one or more of the above radiation treatment units.

In accordance with another aspect of the presently disclosed subject matter there is provided a method for using one or more of the above radiation treatment unit, comprising passing through the pipe the fluid to be treated while exposing it to the radiation intensity within the range of 25-35 mJ/cm².

In accordance with another aspect of the presently disclosed subject matter there is provided a radiation treatment unit including:
a pipe sub-system comprising a pipe for passing therethrough a fluid to be treated, the pipe having an inner diameter, a thickness t of its wall, an outer surface and a smooth inner surface of a diameter and extending between two opposite ends spaced from each other by a distance in a helically coiled manner so that the coiled pipe has a coil inner cylindrical surface defining a cylindrical cavity and a coil outer cylindrical surface of a diameter;
an inner radiation sub-system disposed within the cylindrical cavity and an outer radiation sub-system disposed outside the coil outer cylindrical surface, each comprising at least one inner radiation source extending along a longitudinal axis of the unit;
a unit holding assembly including two end supports and a plurality of outer fixation rods fixedly connected thereto and extending along a longitudinal axis of the system, at a radial distance therefrom greater than that of the radiation sources and the coiled pipe, the end supports receiving therein ends of the radiation sources of both the inner and the outer radiation sub-systems;
the pipe being provided with a spinner therein, the spinner comprising smoothly twisting spinning surfaces spanning between ends thereof. The spinner may be as described above, mutatis mutandis.

All the above mentioned systems can comprise at least one pipe allowing a flow rate of 1000 liter/hour, and has the following parameters: D/D_{C}=0.05, D=11.1 mm.

All the above mentioned systems can comprise at least one pipe allowing a flow rate of 5000 L liter/hour, and has the following parameters: D/D_{C}=0.04, D=22 mm.

All the above mentioned systems can comprise at least one pipe allowing a flow rate of 15,000 liter/hour, and has the following parameters: D/D_{C}=0.04, D=36 mm.

In accordance with another aspect of the presently disclosed subject matter, there is provided a radiation treatment unit comprising:
a pipe sub-system comprising a helically-coiled pipe for passing therethrough a fluid to be treated and defining therein a cylindrical cavity; and
at least one radiation sub-system configured to generate radiation and disposed so as to expose the pipe sub-system to the radiation;
   wherein the pipe is provided with a spinner therewithin, the spinner comprising smoothly twisting spinning surfaces spanning between ends thereof.

The spinning surfaces may be twisted through substantially at least 360° between the ends of the spinner.

The spinning surfaces may span between two helical edges.

The helical edges may tightly fit within the inner cylindrical surface of pipe.

The pitch of the spinning surfaces may be substantially constant between the ends of the spinner.

The pitch of the spinning surfaces may decrease along its length.

The spinner may be made of stainless steel.

The pipe may have a smooth inner surface of a diameter D, the coiled pipe having a coil inner cylindrical surface defining a cylindrical cavity and a coil outer cylindrical surface of a diameter D_{C}, wherein 0.03≦D/D_{C}≦0.1.

The pipe may satisfy the condition D/D_{C}=0.05.

D may be between about 10 mm and about 35 mm.

The pipe may have a linear of between about 1,200 mm and about 1,700 mm.

The pipe may have a wall thickness of between about 0.63 mm and about 1.27 mm.

The pipe may be made of a Fluoropolymer.

The radiation sub-system may comprise at least one radiation source selected from the group including an inner radiation source disposed within the cylindrical cavity and an outer radiation source disposed outside the coil.

The radiation sub-system may comprise the inner radiation source and the outer radiation source.

Each radiation source may extend along the length of the coil.

Each radiation source may comprise one or more ultraviolet lamps.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a front view of the radiation treatment system in accordance with one example of the presently disclosed subject matter;

FIG. 2 is a front view of one radiation treatment unit of the system of FIG. 1;

FIG. 3 is a cross section of the unit of FIG. 2;

FIG. 4A is a right perspective view of the unit of FIGS. 2 and 3;

FIG. 4B is a left perspective view of the unit of FIGS. 2 to 4A;

FIGS. 5 and 6 are enlarged views of the ends of the unit of FIGS. 2 to 4B;

FIG. 7A is a partial perspective view of the coiled pipe of the unit of FIGS. 2 to 6;

FIG. 7B is a cross section of the coiled pipe of FIG. 7A;

FIG. 7C is a cross section taken along line A-A of FIG. 7A;

FIG. 7D illustrates a spinner for use, e.g., with the pipe illustrated in FIGS. 7A through 7C;

FIG. 8 is a front view of the unit of FIG. 2, with the outer lamps and the coil taken out therefrom;

FIG. 9 is a front view of the unit of FIG. 8, with the inner lamps taken out therefrom;

FIG. 10A is a left perspective view of a left flange support of the unit of FIGS. 2 to 9;

FIG. 10B is a right perspective view of a right flange support of the unit of FIGS. 2 to 9;

FIGS. 11A and 11B are left and right perspective views, respectively, of an intermediate support of the unit of FIGS. 2 to 9;

FIG. 12 is a right perspective view of an intermediate central support with coil supporting brackets;

FIGS. 13A to 13N schematically illustrate the assembly of the unit of FIGS. 2 to 12;

FIG. 14 is a right perspective view of a radiation treatment system in accordance with another example of the presently disclosed subject matter;

FIG. 15 is a top view of the radiation treatment system of FIG. 14;

FIG. 16 is a left side view of the radiation treatment system of FIG. 14;

FIG. 17 is a left perspective view of a radiation treatment unit of the system of FIG. 14;

FIG. 18 is a right perspective view of a radiation treatment unit of FIG. 17;

FIG. 19 is a left perspective view of a unit holding assembly of the radiation treatment unit of FIG. 17;

FIG. 20 is a right perspective view of a unit holding assembly of the radiation treatment unit of FIG. 17;

FIG. 21 is a right perspective view of a radiation treatment system in accordance with another example of the presently disclosed subject matter;

FIG. 22 is a right side view of the radiation treatment system of FIG. 21;

FIG. 23 is a left side view of the radiation treatment system of FIG. 21;

FIG. 24 is a right perspective view of the radiation treatment system of FIG. 21, shown without the housing thereof;

FIG. 25 is a top view of the radiation treatment system of FIG. 24;

FIG. 26 is left side perspective view of the radiation treatment system of FIG. 24;

FIG. 27 is an enlarged view of the right end of the radiation treatment system of FIG. 24;

FIG. 28 is a front view of a radiation treatment unit of the system of FIGS. 21 to 27;

FIG. 29A is a top view of a portion of the radiation treatment unit of FIG. 28, showing the sliding elements thereof;

FIG. 29B is an enlarged view of a portion of the sliding element of the radiation treatment unit of FIG. 28;

FIGS. 30 and 31 show a left flange assembly of the radiation treatment unit of FIG. 28;

FIGS. 32A and 32B show a right flange assembly of the radiation treatment unit of FIG. 28;

FIG. 33 shows the lamps and pipes of the radiation treatment unit of FIG. 28;

FIG. 33A is a front view of the radiation treatment unit of FIG. 33 showing only the pipes;

FIG. 33B is a portion of cross section taken along line B-B of FIG. 33;

FIG. 34 shows a right flange assembly of the radiation treatment unit of FIG. 28;

FIGS. 35 and 36 show an inner and an outer support assemblies of the radiation treatment unit of FIG. 28, respectively; and

FIG. 37 is a perspective view of the radiation treatment unit of FIG. 28, showing an axial motion of the outer pipe assembly with respect to the inner pipe assembly.

### DETAILED DESCRIPTION OF EMBODIMENTS

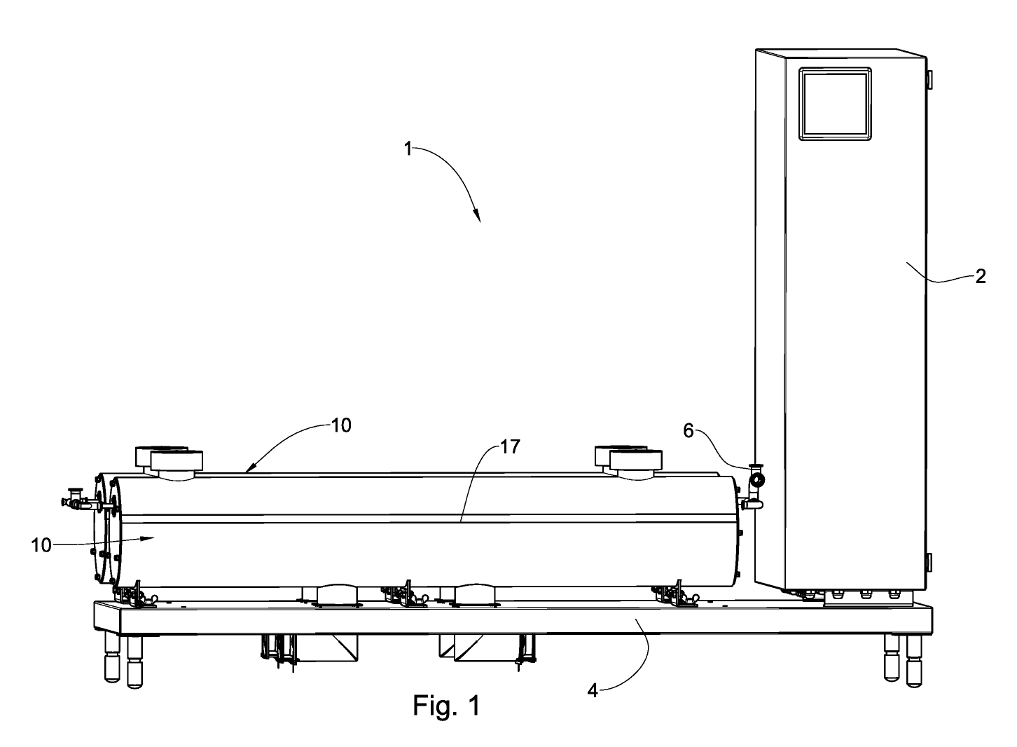

With reference to FIG. 1, there is shown a radiation treatment system 1 in accordance with one example of the presently disclosed subject matter, for treating fluids by exposing them to UV radiation, such as milk, juices, etc.

The system 1 comprises a plurality of UV treatment units 10 each mounted within a housing 17, a control board 2, which is in fluid communication with the units 10 by means of a faucet 6, and a mounting stage 4, to which the units 10 and the control board are fixedly mounted.

The system 1 can be connected to a control system for controlling the system operation, and other systems and/or elements generally known in the art, such as pump balance tank, which are not shown and will not be described in the present application.

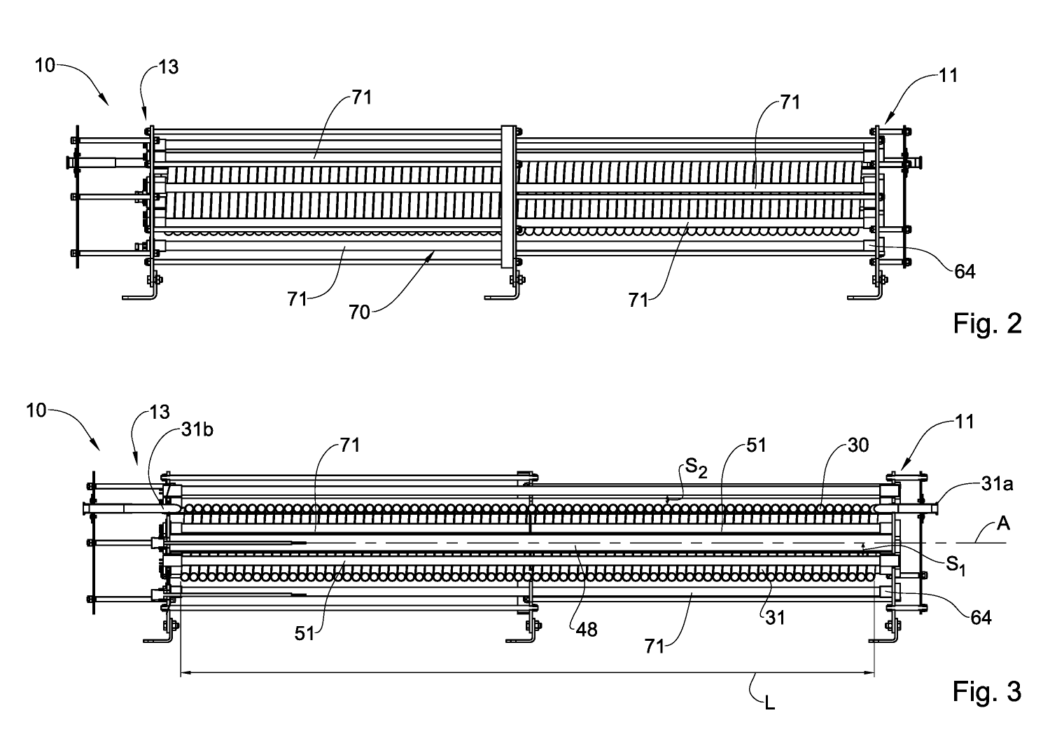

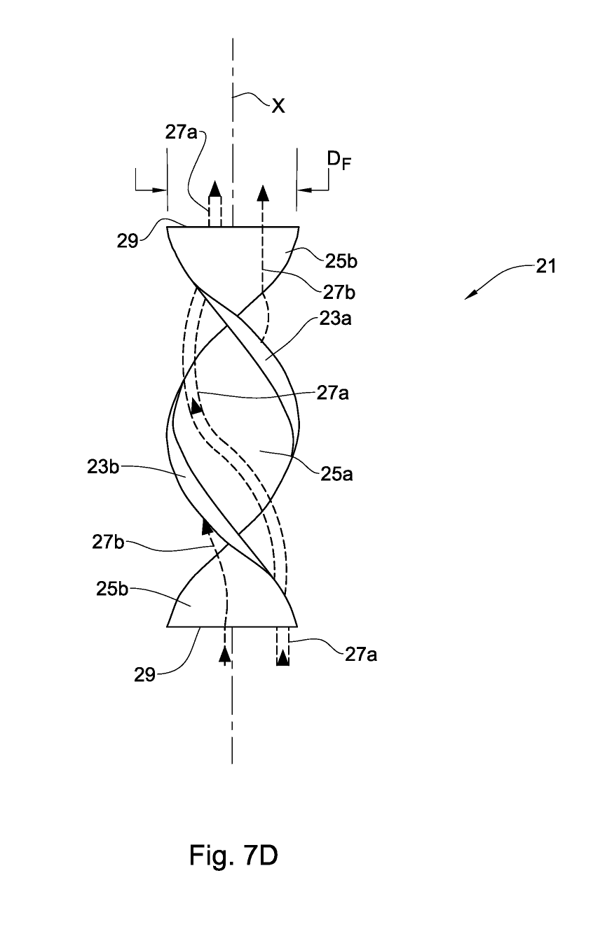

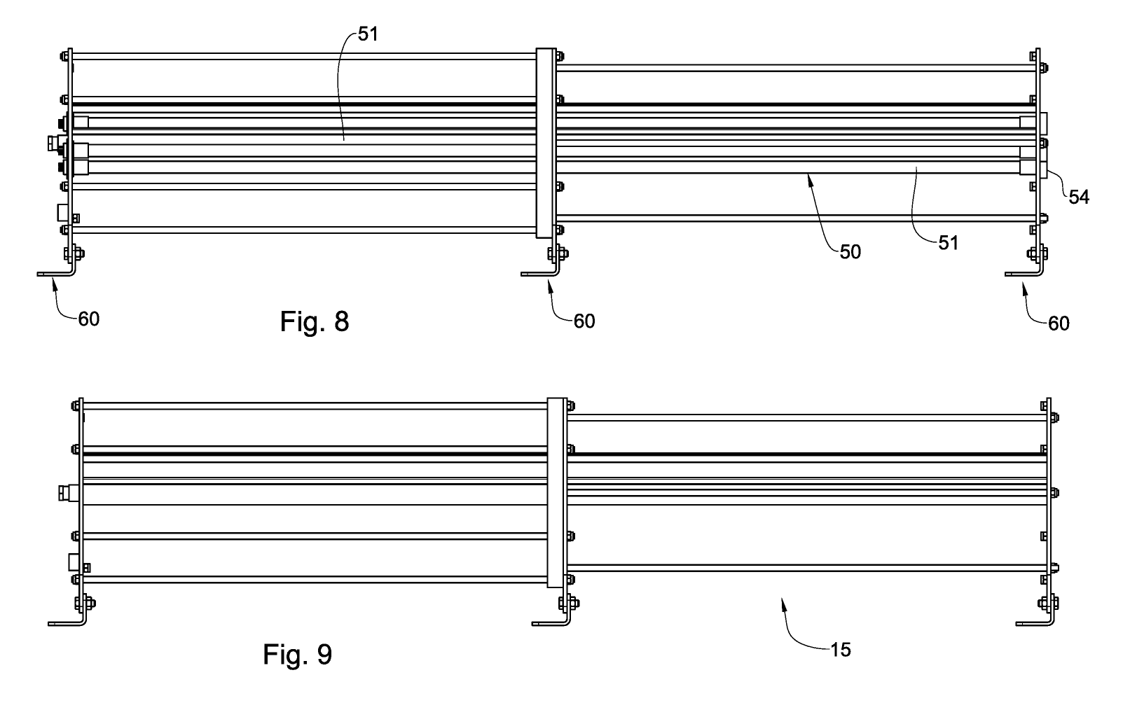

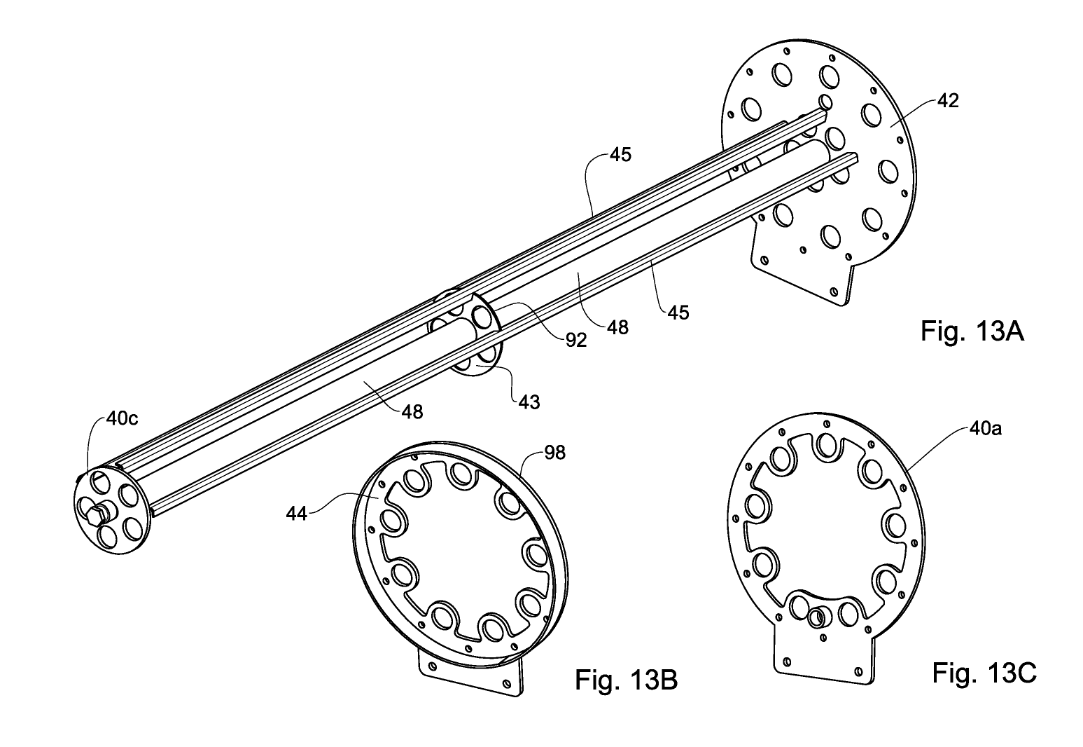

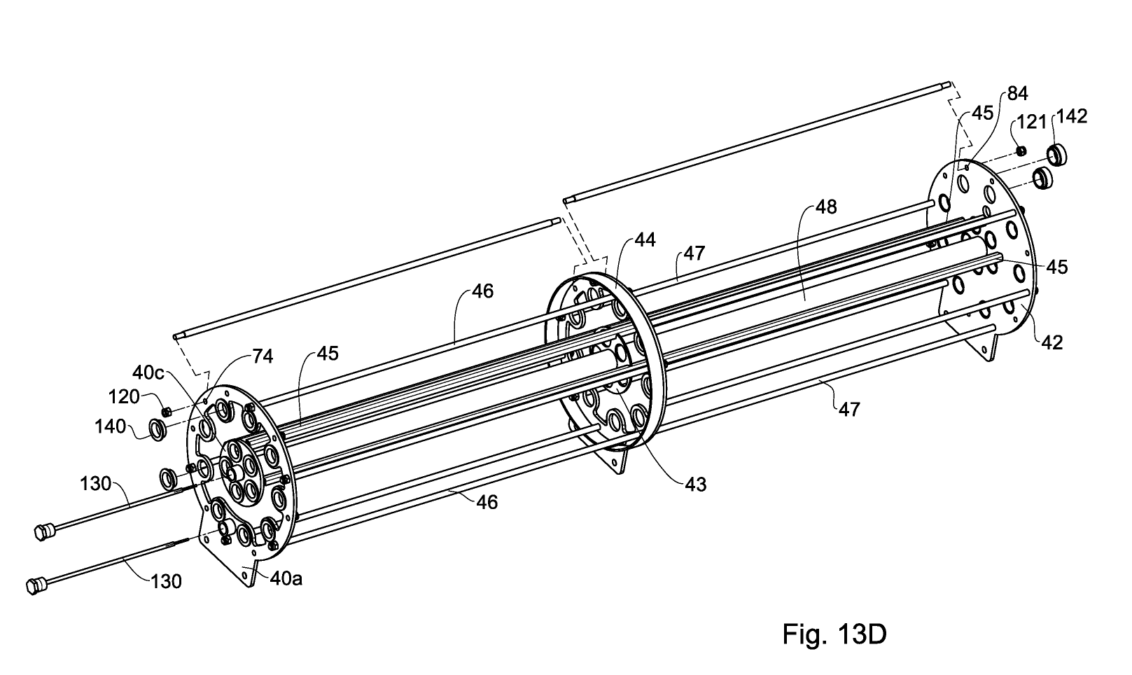

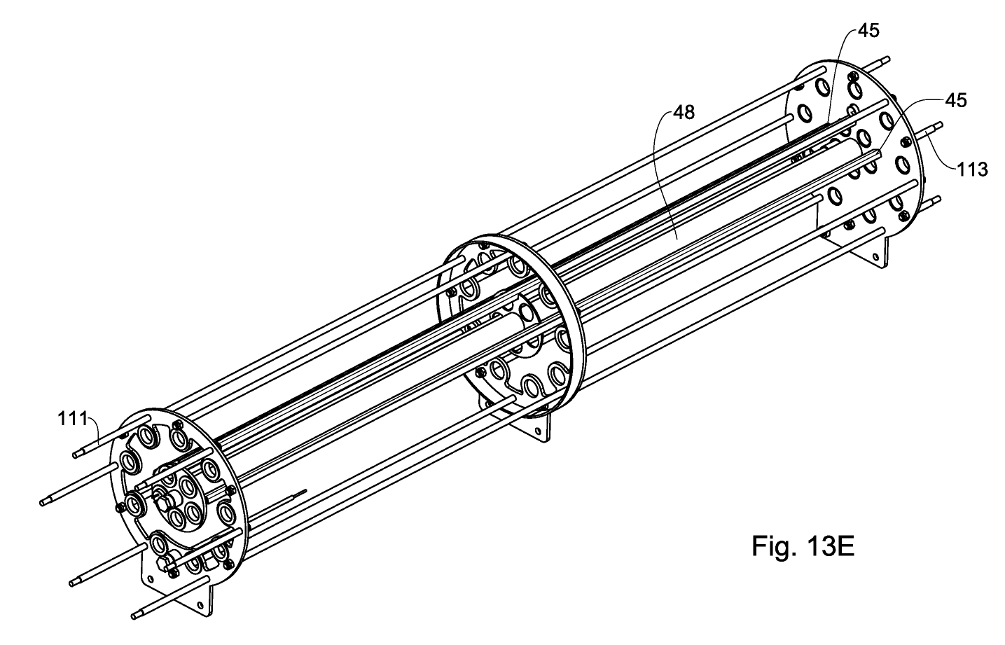

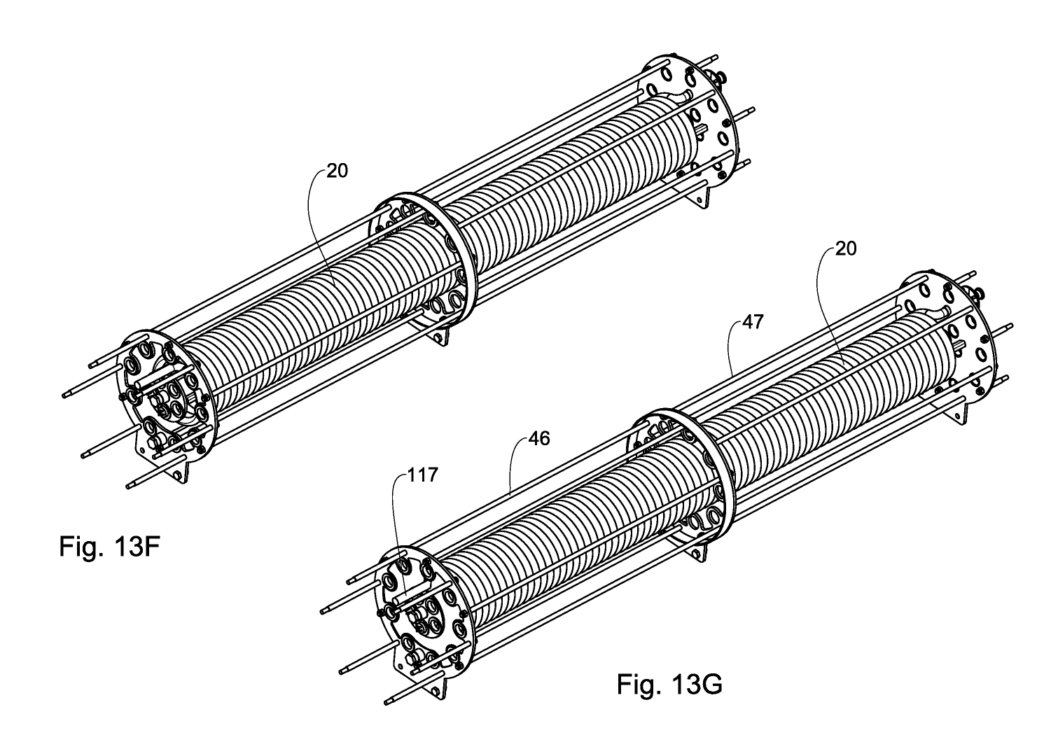

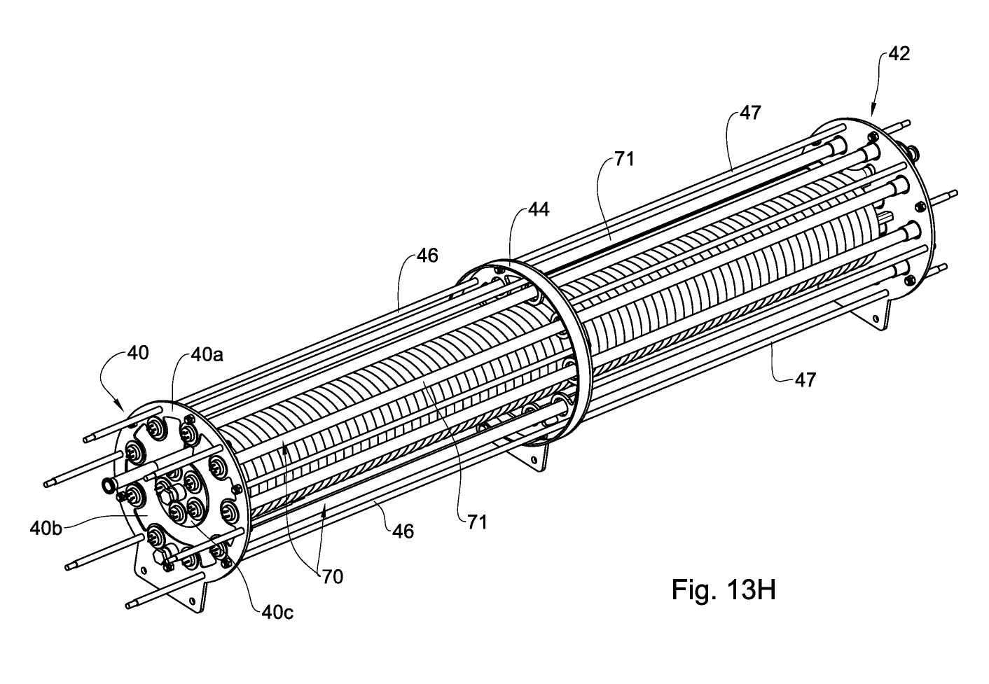

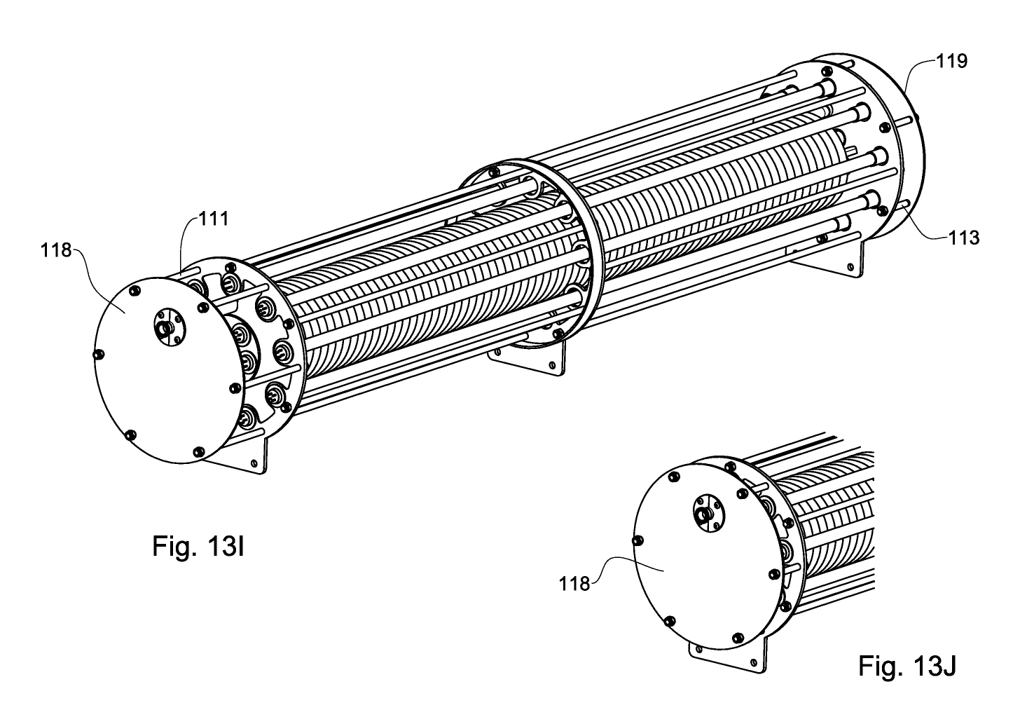

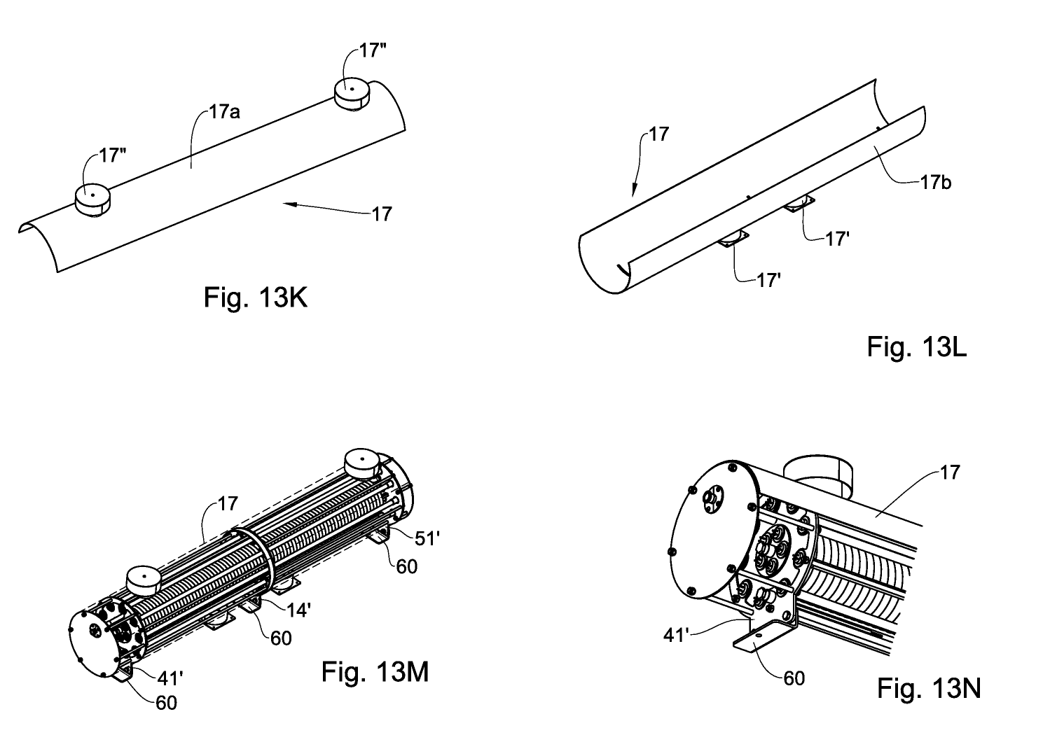

With reference to FIGS. 2 and 3, each radiation treatment unit 10 in the described example is of an elongated shape having a unit central axis A (FIG. 3), and it comprises four main sub-systems as follows:
a coiled pipe sub-system 30 shown within the system in FIG. 3 and shown separately in FIGS. 7A and 7B (for the purpose of simplification this pipe is shown schematically as a cylindrical body in the remaining figures showing the system),
an outer UV radiation sub-system 70 (FIGS. 2 and 13H);
an inner UV radiation sub-system 50 (FIG. 8); and
a unit holding assembly 15 (best seen in FIG. 9), configured for supporting the sub-systems 30, 50 and 70.

All these sub-systems extend between two opposite ends 11 and 13 (FIGS. 2 to 4B) of the elongated radiation treatment unit 10 and are disposed concentrically about the unit central axis A, the arrangement being such that the pipe sub-system 30 surrounds the inner radiation sub-system 50 and is surrounded by the outer radiation sub-system 70, to allow the fluid, which passes through the pipe sub-system, to be exposed to radiation from the inside and the outside. However, a non-concentric arrangement is also possible.

The elements of each sub-system are described below in detail.

The pipe sub-system 30 comprises a helically coiled pipe 31 (FIGS. 7A to 7C), having a pipe wall 33, an inner pipe surface 37, an outer pipe surface 35 and inner and outer diameters Dᵢₙ and Dₒᵤₜ, respectively (FIG. 7C). The inner pipe surface 37 is a smooth surface defining an interior 39 of the pipe 31, for passing therethrough the fluid to be treated between a pipe inlet 31a, which is in fluid communication with the control board 2 via the faucet 6 and a pipe outlet 31b (FIG. 3).

The pipe is made of a material that is highly resistible to UV radiation, has sufficient UV transmission properties, is resistant to high pressures, is qualified for use in contact with food, has a low coefficient of friction with liquids, is bendable, and is resistible to breakage or flattening. One example for such material is Fluorinated Ethylene Propylene (FEP).

The pipe has inner and outer diameters Dᵢₙ and Dₒᵤₜ, and the wall thickness t_{w}. The wall thickness t_{w} of the pipe 31 (FIG. 7C) can be in the range 0.6-1.3 mm, more particularly 0.63-1.27 mm, to allow the pipe to bear relatively large pressure differences between the inlet 31a and the outlet 31b of the pipe. The inner diameter Dᵢₙ of the pipe 31 can be in the range of 10-35 mm, allowing a flow velocity to be in the range of 2-3.5 m/sec.

Experiments show that, for example, the wall thickness of 0.81 mm allows the pipe to bear the pressure differences of up to 12 bar, and the inner diameter of the pipe of 11.1 mm allows the flow velocity of 2.8 m/sec.

As shown in FIG. 7A, the helically coiled pipe 31 (referred to hereinafter: coil 20) has a form of a sleeve with an outer cylindrical surface 34 and an inner cylindrical surface 24 defining a cavity 32, all extending between pipe inlet and outlet 31a and 31b (FIG. 3).

The coil length L (FIG. 3) is selected to correspond to the length of the outer and inner UV radiation sub-systems, and it can be in the range of 1,200-1,700 mm. One example of this length is 1,475 mm, which corresponds to the length of UV lamps used in the outer and inner UV radiation sub-systems described below in more detail.

The number of turns 21 (FIG. 7B) forming the coil 20 influences the time of exposure of the fluid to the UV radiation and thus the efficiency of the treatment process, and therefore it is desired to increase it as much as possible, taking into consideration other parameters such as the pipe length, spacing between the turns, pressure drop, organoleptic properties of the product, etc. The number of turns of the coil in the described example of the system can be in the range of 100-130, and specifically 116.

In the described example of the system, the coil outer diameter Dc can be in the range of 100-150 mm, and specifically 133 mm, and the inner diameter of the pipe Dᵢₙ and the diameter D_{C} of the coil can be chosen so as to satisfy Dean ratio, as explained above, i.e. 0.03≦Dᵢₙ/D_{C}≦0.1, and specifically 0.04 or 0.05.

A spinner, which is generally indicated at 21 in FIG. 7D, may be provided within the pipe 31. It may be located adjacent (i.e., at or near) the inlet 31a thereof, or adjacent (i.e., at or near) the beginning (i.e., most upstream) of the coiled section thereof. In addition, one or more spinners may be disposed along the length of the pipe 31, for example disposed at regular intervals spaced evenly throughout. The spinner 21 comprises a twisted strip having two substantially helical edges 23a, 23b (collectively referred to herein with reference numeral 23) twisting about spinner axis X, and smoothly twisting spinning surfaces 25a, 25b (collectively referred to herein with reference numeral 25) spanning therebetween. Each of the spinning surfaces 25 defines a substantially spiral path therearound, indicated by arrows 27a, 27b (with one being shown with a double line to distinguish it from the other) between ends 29 of the spinner 21. The diameter of the spinner Dₛ may be such that is fits tightly within the pipe 31 (i.e., ≈Dᵢₙ), such that all liquid which flows through the pipe is subject to spinning by the spinner. The axial length of the spinner 21 (i.e., between end 29) may be much less than the length of the pipe 31. For example, it may no more than 1% of the length of the pipe 31. According to some examples, the axial length of the spinner 21 may be no more than about 1/150^{th} of the length of the pipe 31.

It will be appreciated that the term “helical” when used herein the specification and claims in connection with the spinner 21 is not to be limited to shape which meets the strict mathematical definition of a helix. Rather, it is to include any shape which spirals (e.g., corkscrews) about the axis X.

The spinning surfaces 25 are considered to be “smooth” in that they are continuous, i.e., without abrupt changes in direction or angle with respect to the axis X. For example, the spinner 21 may be constructed such that any segment thereof along the axis X is substantially similar to the segments adjacent thereto. Accordingly, fluids which flow about the spinner 21 are imparted with a rotation about axis X, without being subject to unnecessary mixing owing to the geometry of the spinning surfaces 25.

The spinner 21 may be provided such that it is twisted through 360° (i.e., the edges 23 rotate through 360° about axis X) as illustrated in FIG. 7D, or any other suitable construction, including, but not limited to, twisted through 180°, 270°, 540 720°, or any other suitable amount. In addition, it pitch may change smoothly along the length of the spinner 21. For example, the pitch may decrease (i.e., the helix becomes “tighter” along its length) along the length of the spinner 21, and inserted into the pipe 31 such that the portion thereof with the lower pitch is disposed further downstream, such that the speed at which the liquid spins gradually increases as it is introduced into the pipe along the spinner.

The spinner 21 may be made of any suitable rigid material. The material may be highly resistible to UV radiation, be qualified for use in contact with food, and have a low coefficient of friction with liquids. It may have high UV transmission properties, but this is not critical as the spinner 21 is only disposed along a small portion of the length of the pipe 31. According to some examples, it is made of the same material as the pipe 31. According to other examples, it is made from stainless steel.

It has been found experimentally that UV treatment of fluids, e.g., such as in a radiation treatment system 1 as described herein comprising spinners 21 as described above is accelerated compared with UV treatment in such a system in which static mixers (having surfaces which are not smooth thereby introducing mixing along their lengths, e.g., abruptly switching direction, comprising two or more helical sections whose ends are adjacent and not aligned with one another) are used, and compared with UV treatment in such a system in which no spinner or mixers are used. It will be appreciated that in this context, an accelerated treatment is one in which fewer colony-forming units of pathogenic and/or spoilage microorganisms remain compared to a baseline treatment (here, those in which a static mixer or no spinner/mixer is used) when all other parameters are held constant.

It will be appreciated that while the spinner 21 is described herein as constituting a part of the disclosed radiation treatment systems, it may be used within a pipe of a radiation treatment system which does not otherwise fall within the scope of the presently disclosed subject matter, mutatis mutandis.

The UV radiation sub-systems 50 and 70 comprise a plurality of UV tube-shaped inner lamps 51 (FIG. 8) and outer lamps 71 (FIGS. 2 and 3), extending between the unit ends 11 and 13. The UV lamps 51 of the inner sub-system 50 can be identical to the UV lamps of the outer sub-system 70, or can differ therefrom by some of their properties.

All the lamps in accordance with one example of the presently disclosed system have a length in the range of 1,200-1,700 mm, and specifically 1,554 mm (total length) and 1475 (the emitting section) and intensity in the range of 500-1,200 μW/cm².

The lamps 51 of the inner radiation sub-system 50 are disposed within the coil cylindrical cavity 32 (FIG. 7A), being uniformly distributed around the axis A (FIG. 3) and spaced from the coil inner cylindrical surface 24 by a distance s₁. The lamps 71 of the outer radiation sub-system 70 are also uniformly distributed about the axis A but they are spaced from the coil outer cylindrical face 34 by a distance s₂ (FIG. 3), which is substantially equal to s1. The number of inner lamps 51 is smaller than the number of outer lamps 71. In accordance with one particular example, the inner radiation sub-system 50 comprises five lamps and the outer radiation subsystem comprises nine lamps.

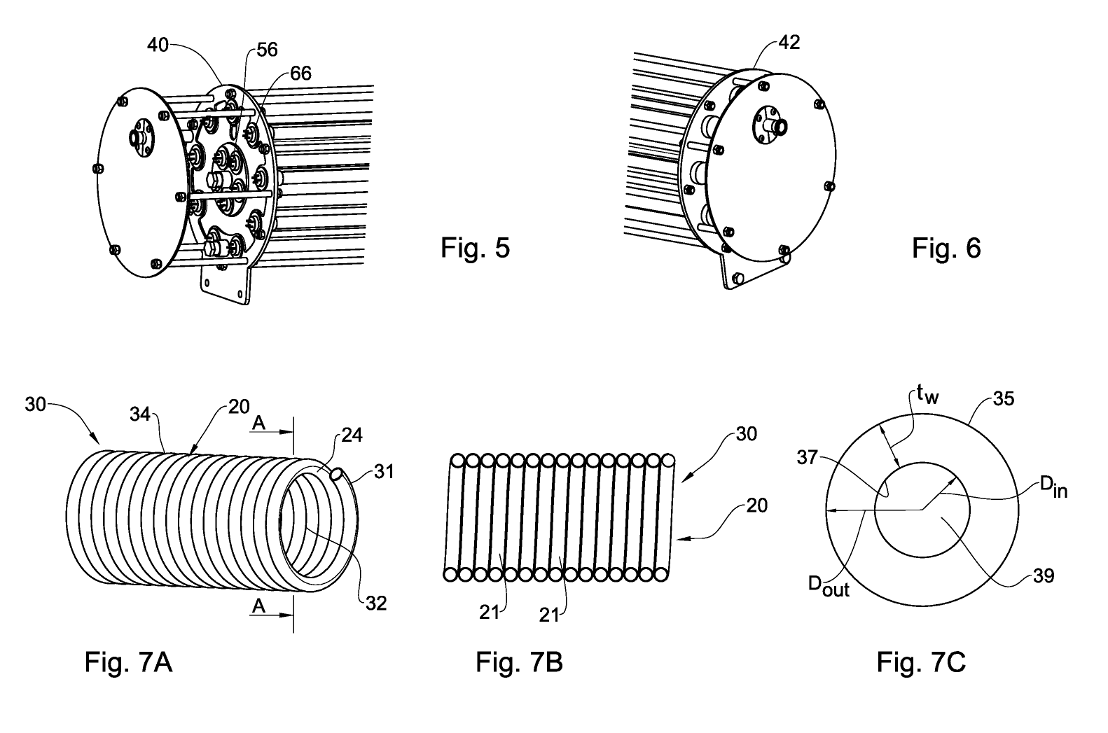

The lamps 51 and 71 are fitted within respective supporting sockets 54 (FIG. 8) and 64 (FIG. 2), which are provided with electrical plugs 56 and 66 (FIG. 5) connecting the lamps to an electrical source (not shown).

As further described in detail, each lamp of the sub-systems 50 and 70 is mounted separately to the unit holding assembly 15.

It should be appreciated the inner and outer radiation sub-systems can each have a single radiation source, for example, an elongated cylindrical radiation source having dimensions suitable for being inserted within the coil cylindrical cavity 32 or an elongated tubular source for surrounding the pipe sub-system.

It should also be appreciated that the inner and outer radiation sub-systems can have identical or different properties, such as light intensity, dimensions, etc.

Referring now to FIGS. 9 to 12, there is shown the unit holding assembly 15 configured for holding the pipe sub-system 30, the inner radiation sub-system 50 and the outer radiation sub-system 70.

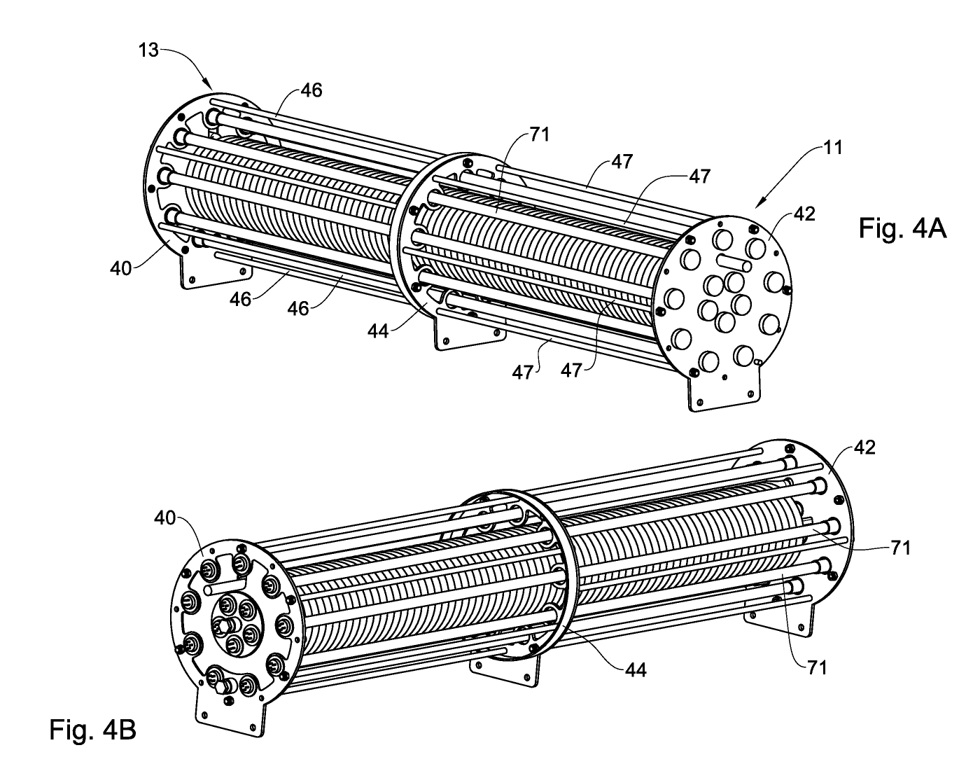

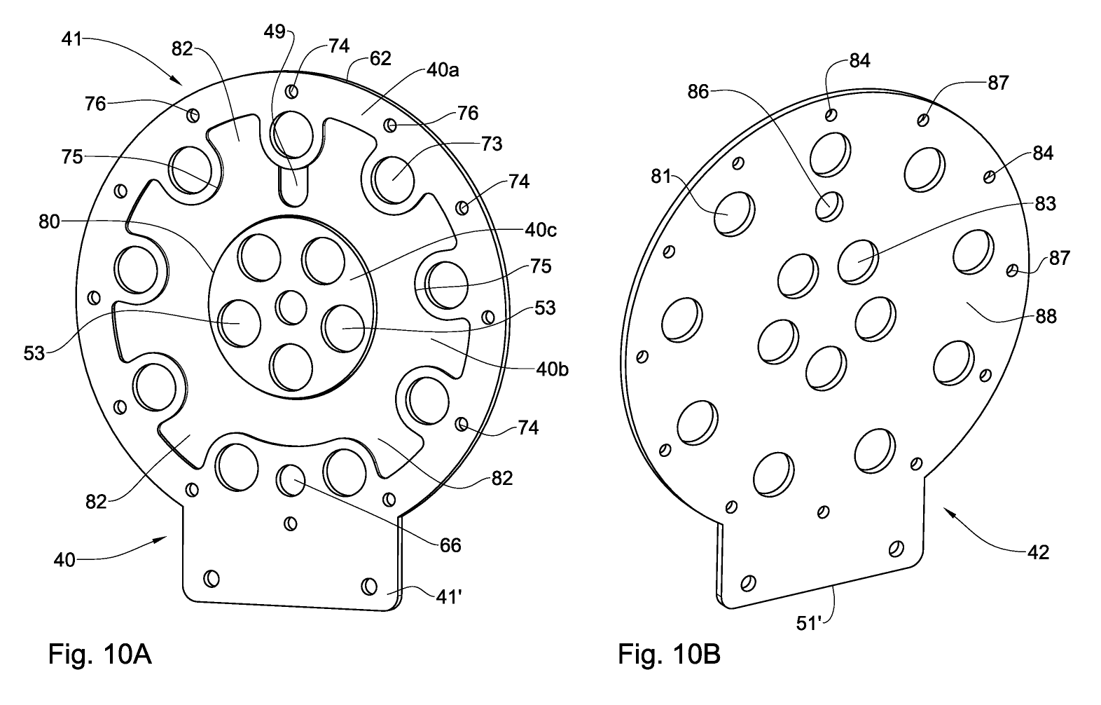

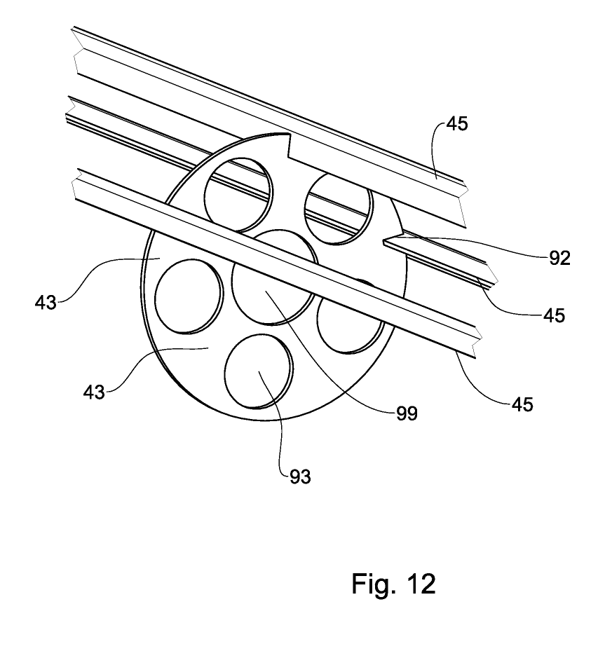

The unit holding assembly 15 is in the form of a structure comprising a number of vertically oriented supports for supporting the coiled pipe and the UV lamps, and a plurality of horizontally oriented fixation rods connecting the supports, one rod—extending coaxially with the assembly central axis A, and others—extending at a radial distance therefrom, for providing the unit holding assembly 15 with a structural integrity. In particular, the unit holding assembly 15 comprises:
a left flange support 40 (FIG. 10A), a right flange support 42 (FIG. 10B), an intermediate central support 43 and an intermediate lateral support 44 (all are also seen in FIGS. 13E to 13H);
an outer fixation assembly comprising a plurality of left fixation rods 46 and right fixation rods 47 extending between the corresponding left and right supports 40 and 42 and the intermediate lateral support 44 (seen in FIGS. 4A and 4B); and
an inner fixation assembly comprising a central fixation rod 48 (FIGS. 3, 13A, 13D and 13E) passing through the intermediate central support 43, and a plurality of coil supporting brackets 45 (best seen in FIGS. 12, 13A, 13D and 13E) connected to and extending outwardly from the an intermediate central support 43.

The left and right flange supports 40 and 42 are disposed at the ends 11 and 13 of the radiation treatment unit 10 and the intermediate lateral support 44 with the intermediate central support 43 being disposed therebetween, and the two intermediate supports 44 and 43 being aligned with each other along a plane perpendicular to the central axis A.

The supports 40, 42 and 44 are fixedly mounted to the mounting stage 4, as shown in FIG. 1, by means of mounting assemblies 60

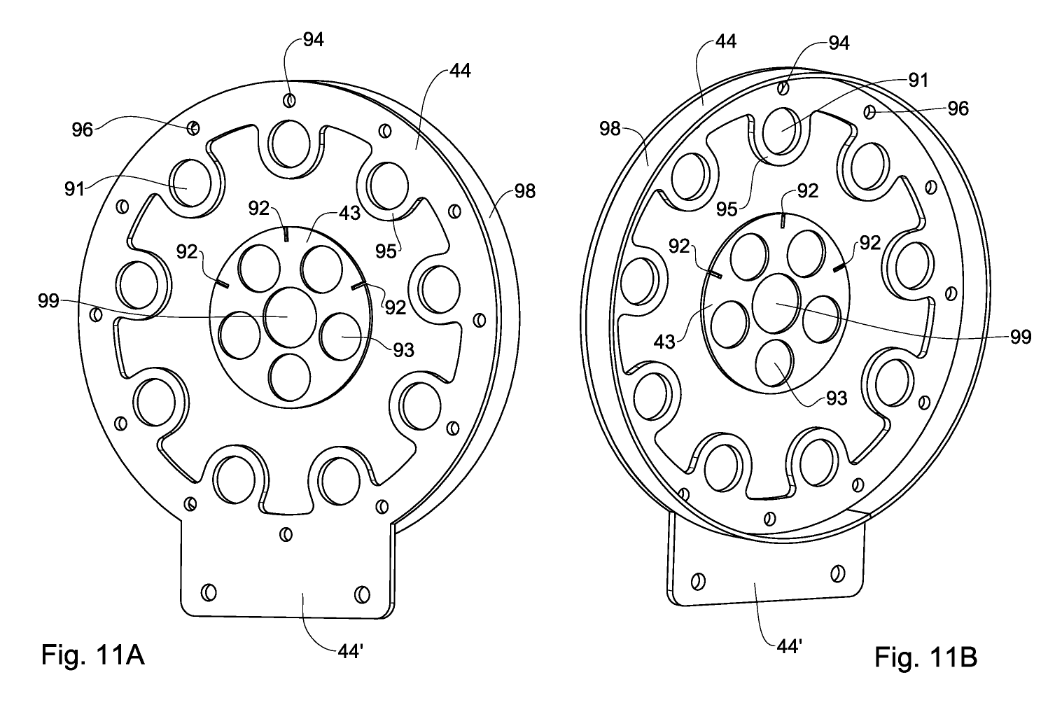

(FIG. 8), thereby fixing the radiation treatment unit 10 to the stage 4. With reference to FIGS. 10A to 11B, the supports 40, 42 and 44 all having bases 41′, 51′ and 44′ for the fixation thereof to the mounting stage 4 by means of the mounting assemblies 60.

The body 41 of the left flange support 40 is assembled of three body portions: a lateral portion 40a, a central portion 40c, and a complementary portion 40b.

With reference to FIG. 10A, the lateral portion 40a is formed with:
rod openings 74 disposed at a maximal radial distance from the axis A adjacent an outer edge 62 of the lateral portion and circumferentially spaced from each other, for receiving therein left ends 46′ of the left outer supporting rods 46;
outer lamp openings 73 disposed at locations angularly spaced from, and radially closer to the central axis A than the rod openings 74, the lamp openings 73 being distributed with respect to the axis A and being configured for receiving therein the left ends 71′ of the outer lamps 71;
a bottom opening 66 for temperature sensor.

The outer portion 40a can be further formed with mounting holes 76 disposed at locations radially and/or angularly spaced from the rod and lamp openings, for attachment thereto of external elements such as spacers 111 (FIG. 13E).

In the present example, the lateral portion 40a has a generally annular disk shape with a number of loop-like protrusions 75 incorporating the lamp openings 73.

The central portion 40c is formed with inner lamp openings 53 circumferentially distributed about the axis A, for receiving therein left ends 51′ of the inner lamps 51, and a central opening 72 configured to receive therein the left end of the central fixation rod 48.

The complementary portion 40b is detachably mounted between the lateral portion 40a and the central portion 40c, and is formed with an opening 49 for allowing passage therethrough of an outlet nozzle 115 in such a manner that it can project outwardly from the system and thereby provide fluid communication between the outlet 31b of the coiled pipe 20 and an accumulation system or other system at the exterior of the system, where treated fluid can be accumulated/received.

In the present example, the central portion 40c has a round disk shape and the complementary portion 40b is in the form of a shaped disc with a central aperture 80 configured to receive therein the central portion 40c and a number of fin-like protrusions 82 configured to occupy all the space between the loop-like protractions 75.

With reference to FIG. 10B, the right flange 42 is substantially similar to the left flange 40, with exception of it being a unitary body, and it comprises rod openings 84, outer lamp openings 81, inner lamp openings 83, inlet nozzle opening 86 and right spacer openings 87.

With reference to FIGS. 11A and 11B, the intermediate lateral support 44 comprises left outer rod openings 94 and right outer rod openings 96 disposed at the same radial distance from, but at different angular locations with respect to, the central axis A, and it comprises loop-like protrusions 95 formed with outer lamp holes 91 for receiving therethrough the outer lamps 71.

The intermediate central support 43 comprises inner lamp holes 93 for receiving therethrough the inner lamps 51, a central hole 99 for receiving therethough the central supporting rod 48, and slots 92 for receiving therein the coil supporting brackets 45 (FIG. 12).

The intermediate lateral support 44 further comprises a rim 98 fixed to the circumference thereof, as shown in FIG. 13B, when the unit is assembled for supporting the half 17a of the housing 17 (FIG. 13K).

Reverting to FIGS. 2, 3, 8 and 9, the structure of the unit holding assembly 15 is such that the central inner supporting rod 48 and the coil supporting brackets 45 extend along the whole length of the unit 10, between the flange supports 40 and 42 and through the intermediate central support 43, while the right supporting rods 46 extend between the right flange support 42 and the intermediate lateral support 44 and the left supporting rods 47 extending between the left flange support 40 and the intermediate lateral support 44.

The intermediate central support 43 and the intermediate lateral support 44 provide an additional support to the lamps and the rods of the unit 10, without adding the complexity of the unit.

The system further comprises temperature sensors 130 (FIG. 13D).

The reference is now being made of FIGS. 13A to 13N showing the process of assembly of the unit 10, as follows:
a. The right flange support 42, the intermediate central support 43, and the central portion 40c of the left flange support 40 are mounted on the central inner supporting rod 48, which is welded to the right flange 42 with this rod passing through their corresponding openings, and the coil supporting brackets 45 are mounted within the slots 92 (FIG. 13A);
b. The intermediate lateral support 44 is fitted with the rim 98 (FIG. 13B) and a lateral portion 40a of the left flange support 40 is provided (FIG. 13C);
c. The left and right supporting rods 46 and 47 are inserted within the rod openings 74 and 84, and fixed by corresponding nuts 120 and 121 (FIG. 13D);
d. Temperature sensors 130 (FIG. 13D) are mounted to the portions 40a and 40c;
e. Plugs 140 and 142 for the outer lamps 71 are mounted in the lamp openings 73 and 81 (FIG. 13D);
f. Left and right spacers 111 and 113 are inserted within the corresponding spacer openings 76, 74 and 84, 87 (FIG. 13E, the openings being shown in FIGS. 10A and 10B);
g. The coil 20 (schematically shown as a pipe) is inserted (FIG. 13F) while being guided by the coil supporting brackets 45;
h. An outlet nozzle 117 is inserted (FIG. 13G);
i. The complementary portion 40b of the flange support 40 is fitted between the portions 40a and 40c (FIG. 13G);
j. The outer and inner lamps 71 and 51 are inserted through the corresponding openings in the flange supports 40 and 42, and intermediate lateral supports 44, and are fixed thereto (FIG. 13H);
k. External protecting plates 118 and 119 are fixed to the spacers 111 and 113 (FIGS. 131 and 13J);
l. The halves 17a, having upper vents 17″ and 17b having fans 17′ of the housing 17 are fitted to cover the whole unit (FIGS. 13K to 13N); and
m. Fixing the bases 41′, 51′ and 44′ to the stage 4 (the stage shown only in FIG. 1) by means of mounting assemblies 60 (FIGS. 13M and 13N).

It should be appreciated that the assembly of the unit 10 is such that it allows easy access, removal and replacement of different elements thereof. For example, the lamps 51 and 71 are mounted directly to the flange supports 40 and 42, which allows an easy access to each of the lamps without disassembling the whole unit.

As detailed above, there are several factors that influence the efficiency of the treatment process that have to be taken into consideration, namely: the intensity of the radiation, the time of the exposure of the fluid to the radiation and turbulence conditions, specifically the conditions that influence the uniformity of the fluid mixing.

A system with the parameters described above, was built and optimal time of exposure when using it was determined experimentally. Thus, the experiments yielded that the exposure time of 17 sec is sufficient for effective treatment of raw milk.

Total intensity of the system was then determined based on the exposure time and the intensity of the lamps. Surprisingly, the total intensity was found to be in the range of 9-15 mJ/cm², and more specifically 12.7 mJ/cm².

As to improving the uniformity of the fluid mixing, as indicated above, the diameters of the coil and the tube were chosen so as to satisfy Dean condition 0.03<D/Dc<0.1. Specifically it was experimentally determined that the most efficient mixing occurs when the ratio D/Dc is equal to 0.04 or 0.05.

A process of designing the system of the presently disclosed subject matter can be summarized by means of the following steps:
a) determining the length of the lamps, taking into consideration that the lamps having greater length will allow a longer exposure time. Thus, the length of the lamps correspond to the desired exposure time range;
b) determining the diameters of the coil and the pipe in accordance with Dean number;
c) determining the length of the pipe and number of turns of the coil;
d) iteratively determining the inner pipe diameter;
e) determining the pressure difference between the ends of the unit, taking into consideration the limitation that the pressure difference does not exceed 12 bar.

To determine the pressure difference of step (e) above, the following equations were used, in which V is a fluid velocity, ρ is a fluid density, f is a friction factor, and e is absolute roughness of the pipe.

$$0.03 \leq D / {D}_{c} \leq 0.1 ;$$

$$Δ P = f ρ {V}^{2} / 200000 \cdot L / D ;$$

$${f}_{s} = {[ \frac{1}{- 2 log ( \frac{\frac{ε}{D}}{3.7} ) + ( \frac{2.51}{Re \sqrt{f}} )} ]}^{2}$$

It should be appreciated that the equations used for the calculation of the friction factor are specifically suitable for coiled pipes, such as the pipe of the presently disclosed subject matter.

In accordance with one specific example, the system was used for passing a fluid with the following parameters: Flow velocity (V)=2.8 m/sec; ρ=1,029 kg/m³; and Re=12,021, while the system had the following parameters:
t_{w}=0.81;
Dᵢₙ=11.1;
L=1,475 mm;
Number of turns of the coil=116;
Dᵢₙ/D_{C}=0.05;
Total lamp length=1,554 mm;
Emission lamp length=1475 mm;
Lamp intensity=1150 μW/cm²

The system having the above parameters provided the exposure time of 25 sec, total intensity of 12.7 mJ/cm² and pressure difference of about 12 bar.

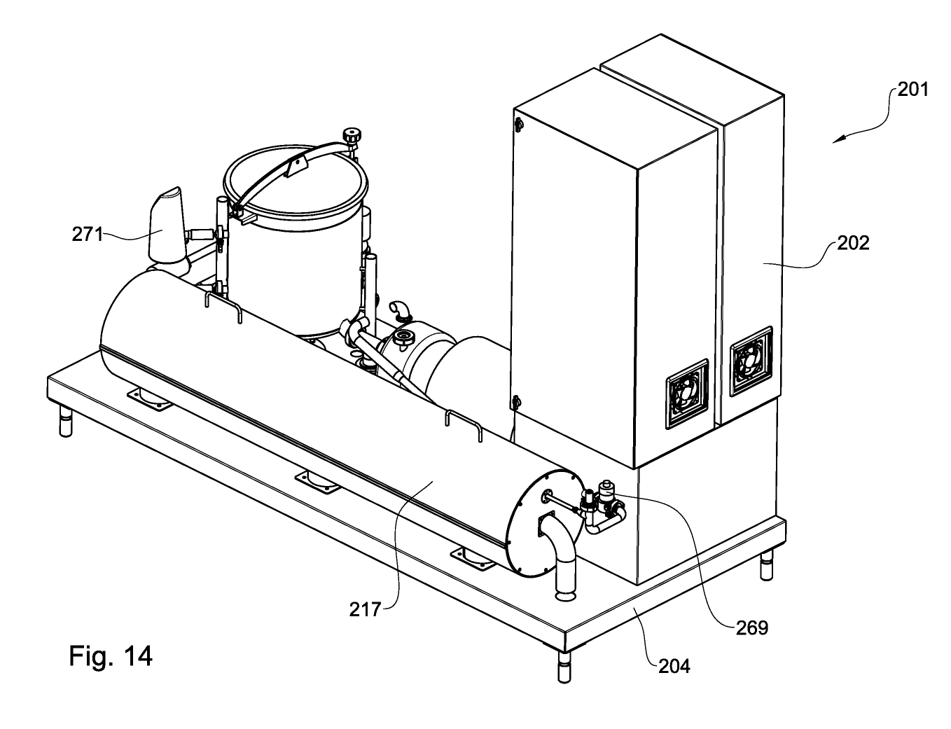

Referring now to FIGS. 14 to 20, there is shown a UV treatment system 201 in accordance with another example of the presently disclosed subject matter.

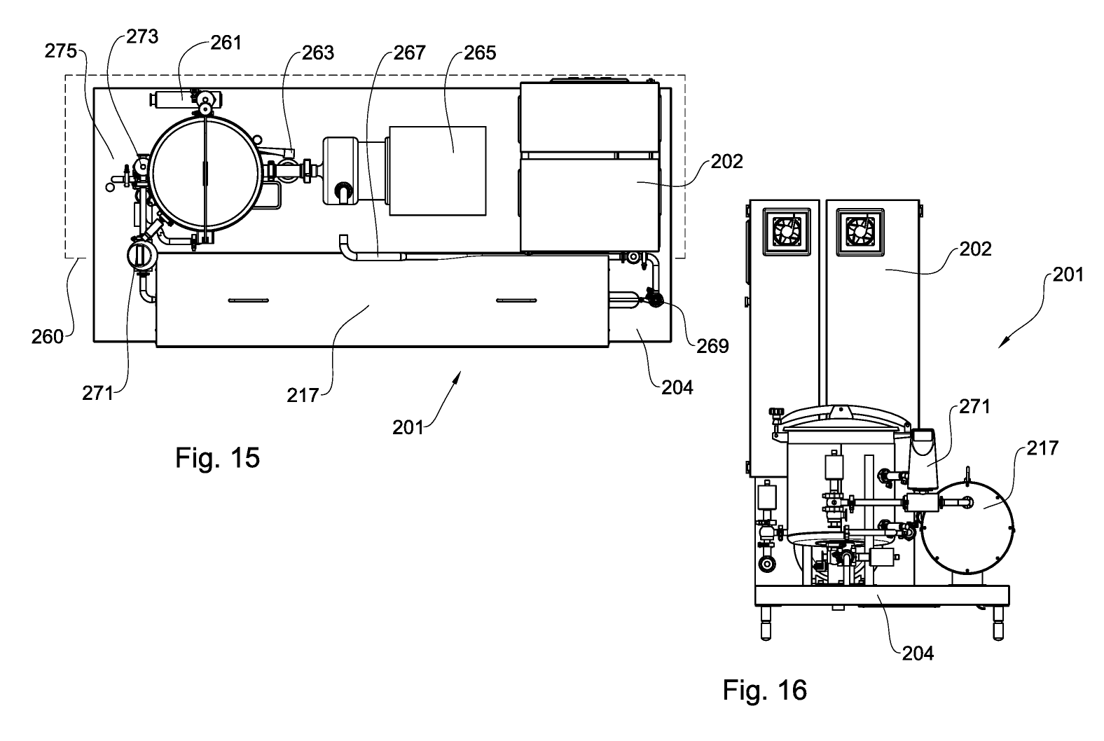

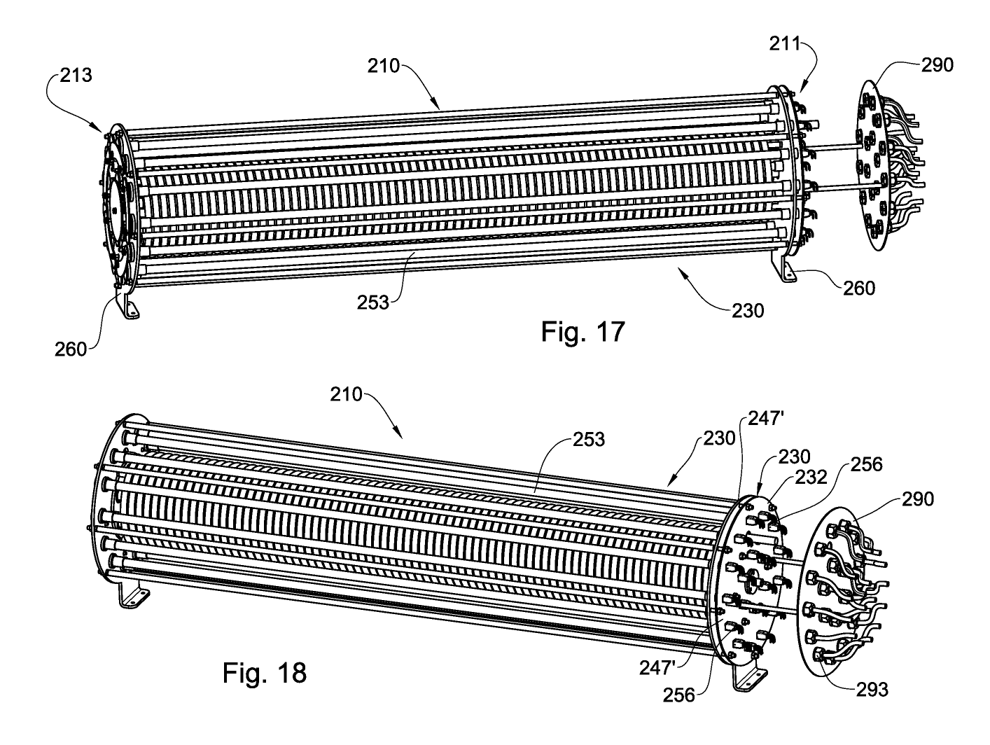

The system 201 comprises a pre/post-treatment system 260 (shown in dotted lines in FIG. 15) and a UV treatment unit 210 (FIGS. 17 and 18) mounted within a housing 217, and a mounting stage 204, to which the elements of the pre/post-treatment system 260 and the unit 210 are fixedly mounted.

The pre/post-treatment system 260 comprises an inlet connected by means of a faucet 261 to a buffer tank 263, a pump 265 which is in fluid communication with the UV treatment unit 210 by means of a pre-treatment pipe 267, the latter being fitted with an inlet flow rate regulating valve 269, outlet flow control sensor 271, and a control board 202, which is in communication with all the elements of the pre/post-treatment system 260 and the UV treatment unit 210.

The pre/post-treatment system 260 further comprises a couple of faucets 273 and 275 disposed between the outlet flow control faucet 271 and the buffer tank 263, responsible for directing the fluid exiting the UV treatment unit 210 to either repeating the treatment, continuing to post-treatment processes or sewage.

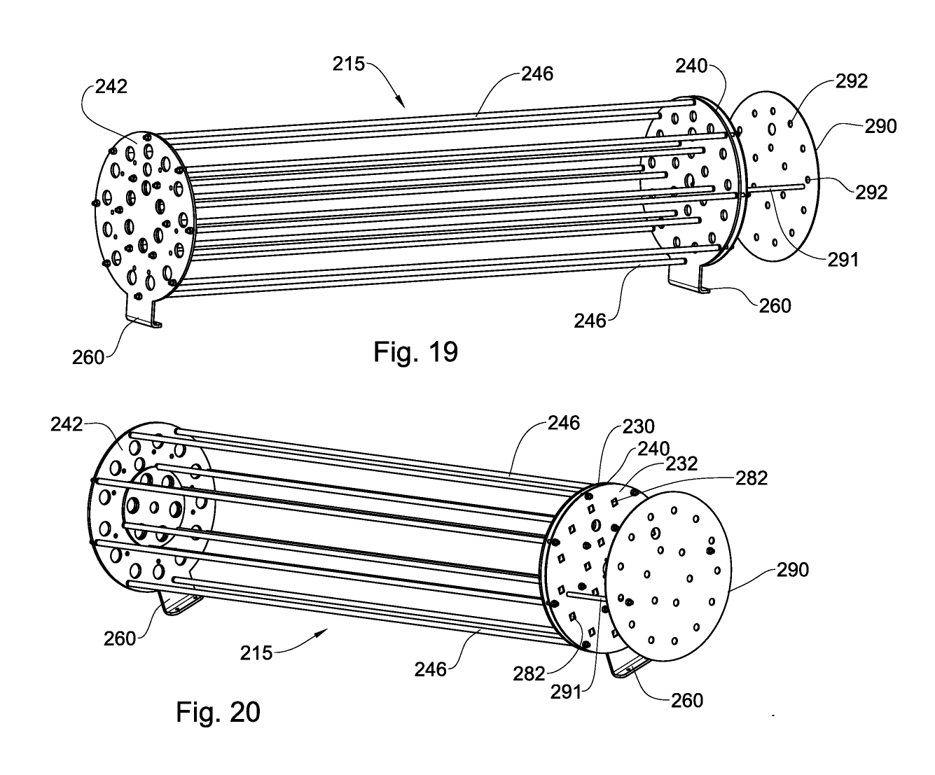

The UV treatment unit 210 comprises a coiled pipe sub-system 230, an inner UV radiation sub-system 250, an outer UV radiation sub-system 270, all similar to the corresponding assemblies of the UV treatment unit 10, and a unit holding assembly 215 (FIGS. 19 and 20), which differs from the unit holding assembly 15 of the UV treatment unit 10, as detailed below.

The unit holding assembly 215 is in the form of a structure comprising vertically oriented supports for supporting the coiled pipe and the UV lamps, and a plurality of horizontally oriented fixation rods extending between the supports.

In particular, the unit holding assembly 215 comprises a first flange support 240, a second flange support 242 and a plurality of fixation rods 246 extending therebetween.

The first and second flange supports 240 and 242 are disposed at the ends 211 and 213 of the radiation treatment unit 210 and are fixedly mounted to the mounting stage 204 by means of mounting portions 260, thereby fixing the UV treatment unit 210 to the stage 204.

The flange support 242 is substantially similar to the flange support 42 of the unit 10, for supporting the rods 246 and inner and outer lamps 251 (not shown) and 253.

The flange support 240 comprises an inner flange 230 and an outer flange 232 slightly spaced apart therefrom.

The arrangement is such that the inner flange 230 supports the supporting sockets 254 (not seen) of the lamps 251 and 253 which pass through the corresponding openings thereof and also supports end portions of the fixation rods 246 which pass through the corresponding openings thereof.

The outer flange 232 comprises openings 282 shaped so as to anchor the electrical plugs 256 (FIG. 18) of the lamps and openings 284 (not seen) shaped so as to anchor the ends 247′ of the rods 246.

The UV treatment unit 210 further comprises a cable flange 290, spaced from the outer flange 232 and supported by a couple of flange support rods 291.

The cable flange 290 comprises a plurality of openings 292 (FIG. 19) for receiving therethrough cables connected to the electrical plugs 256 of the lamps (the portions of the cables extending between the plugs and the cable supports are not shown).

The above structure allows a convenient and organized cable arrangement and prevents a close access to the area of the electrical plugs.

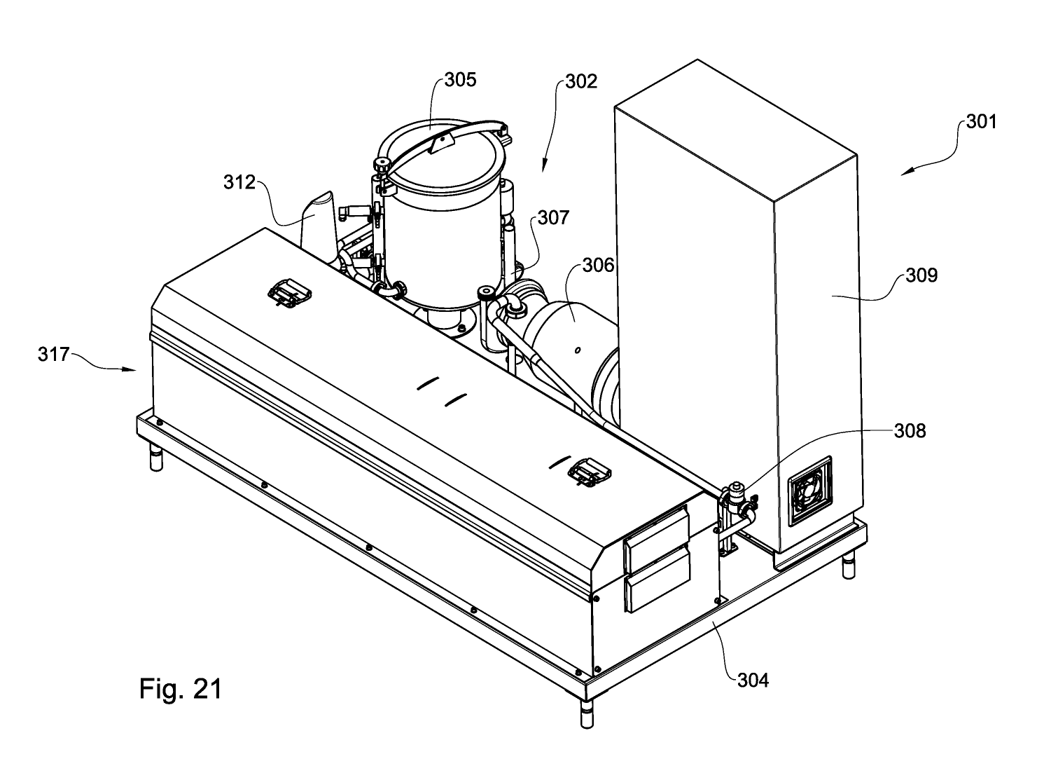

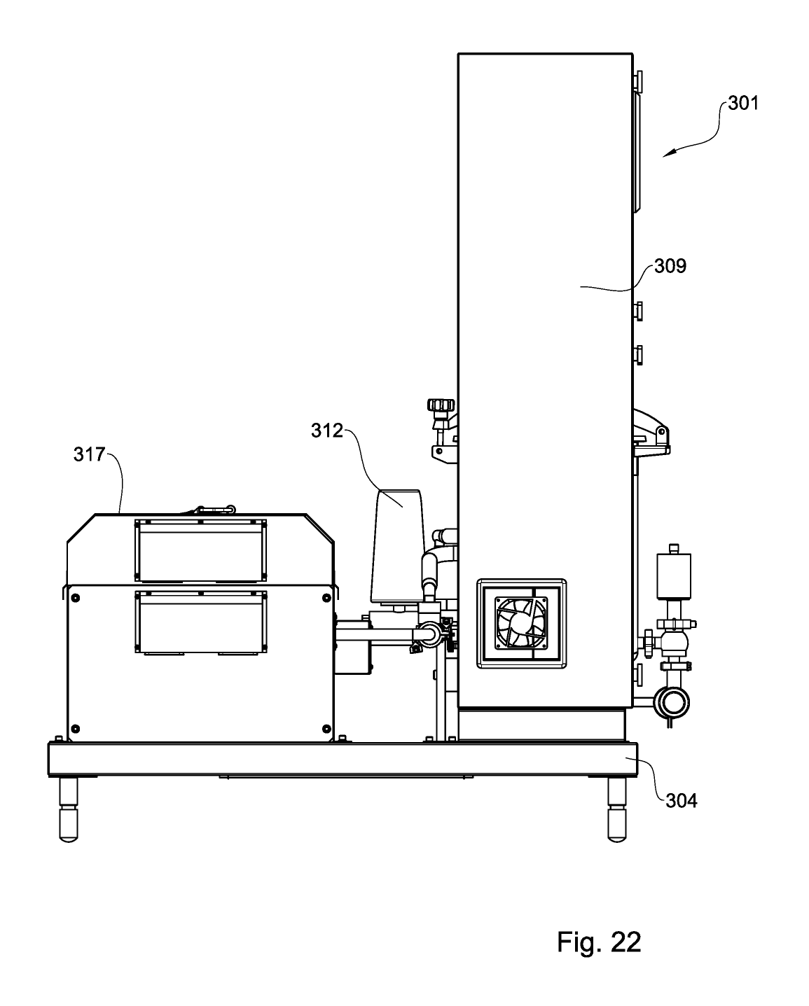

Referring now to FIGS. 21 to 26, there is shown a UV treatment system 301 in accordance with another example of the presently disclosed subject matter.

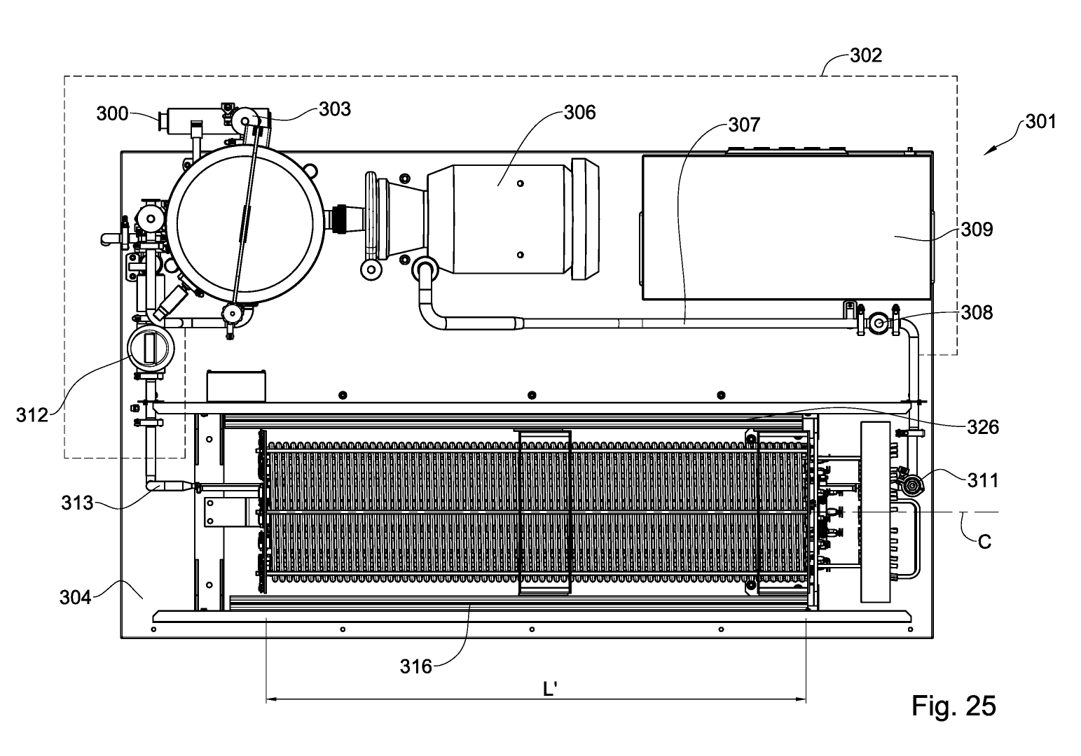

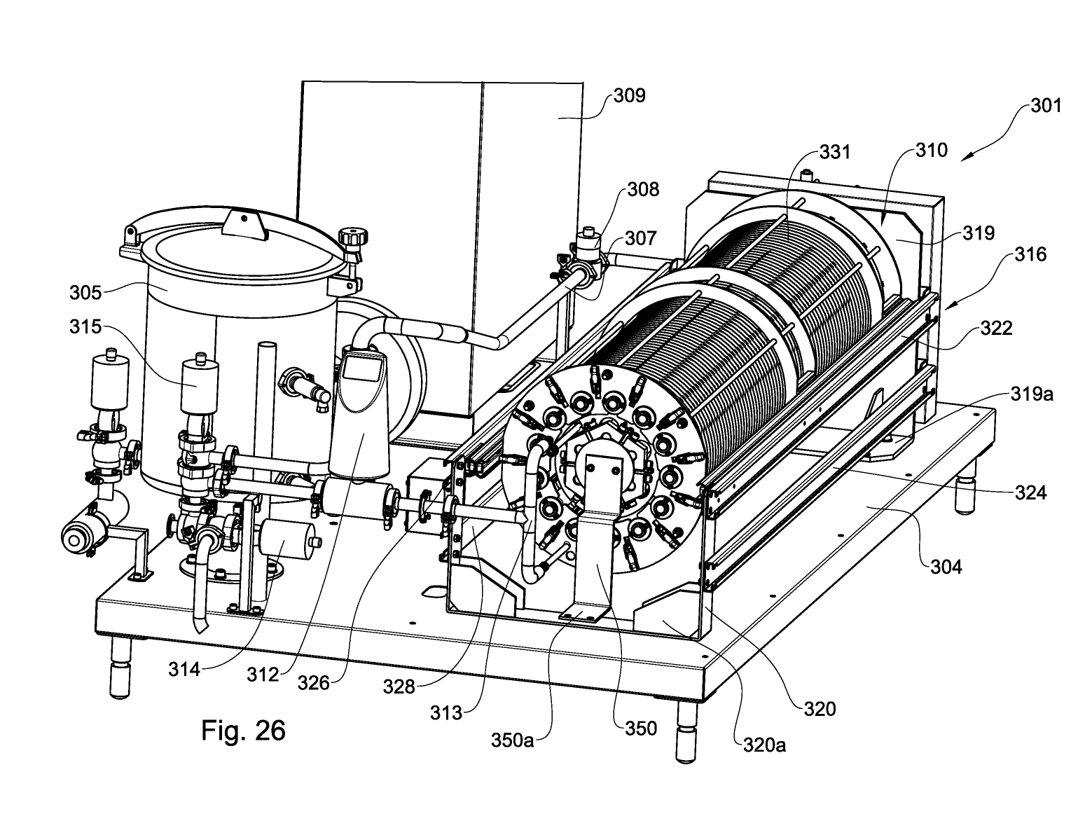

The system 301 comprises a pre/post-treatment system 302 (shown in dotted line in FIG. 25) and a UV treatment unit 310 (FIG. 26) mounted within a housing 317, and a mounting stage 304, to which the elements of the pre/post-treatment system 302 and the unit 310 are fixedly mounted.

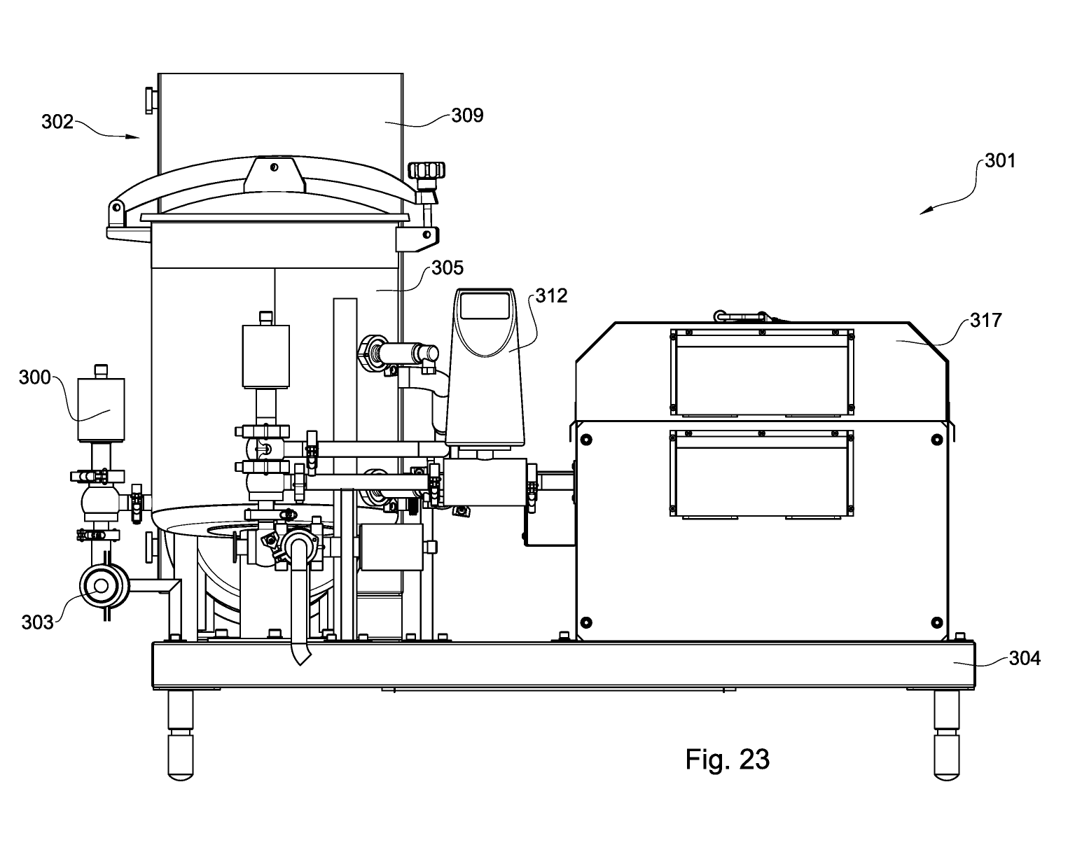

The pre/post-treatment system 302 comprises an inlet 300 (FIGS. 23 and 25) connected by means of a faucet 303 to a buffer tank 305, a pump 306 which is in fluid communication with the UV treatment unit 310 by means of a pre-treatment pipe 307, the latter being fitted with an inlet flow rate regulating valve 308, an outlet flow control sensor 312, and a control board 309, which is in communication with all the elements of the pre/post-treatment system 302 and UV treatment unit 310.

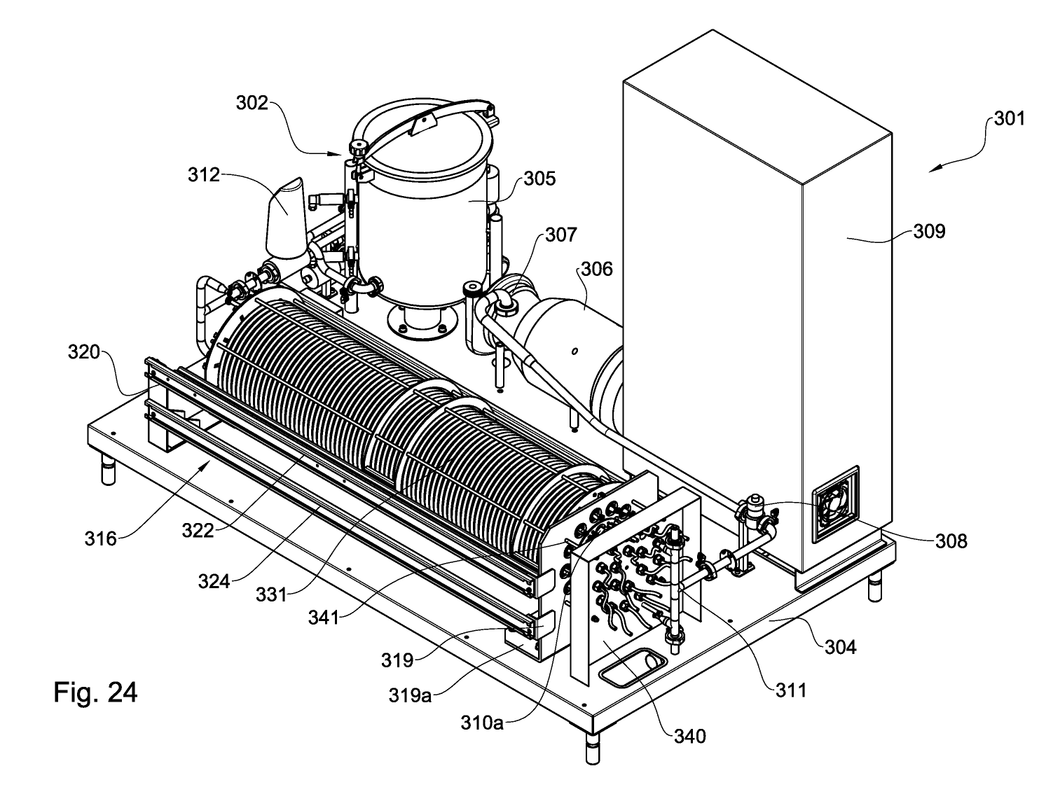

The pre/post-treatment system 302 further comprises several elements disposed within the housing 317 (FIGS. 24 to 27), namely, an inlet distributor valve 311 disposed between the inlet flow rate control faucet 308 and an inlet 310a of the UV treatment unit 310, and an outlet distributor valve 313 (FIGS. 25 and 26) disposed between an outlet 310b of the UV treatment unit 310 and the faucet 312.

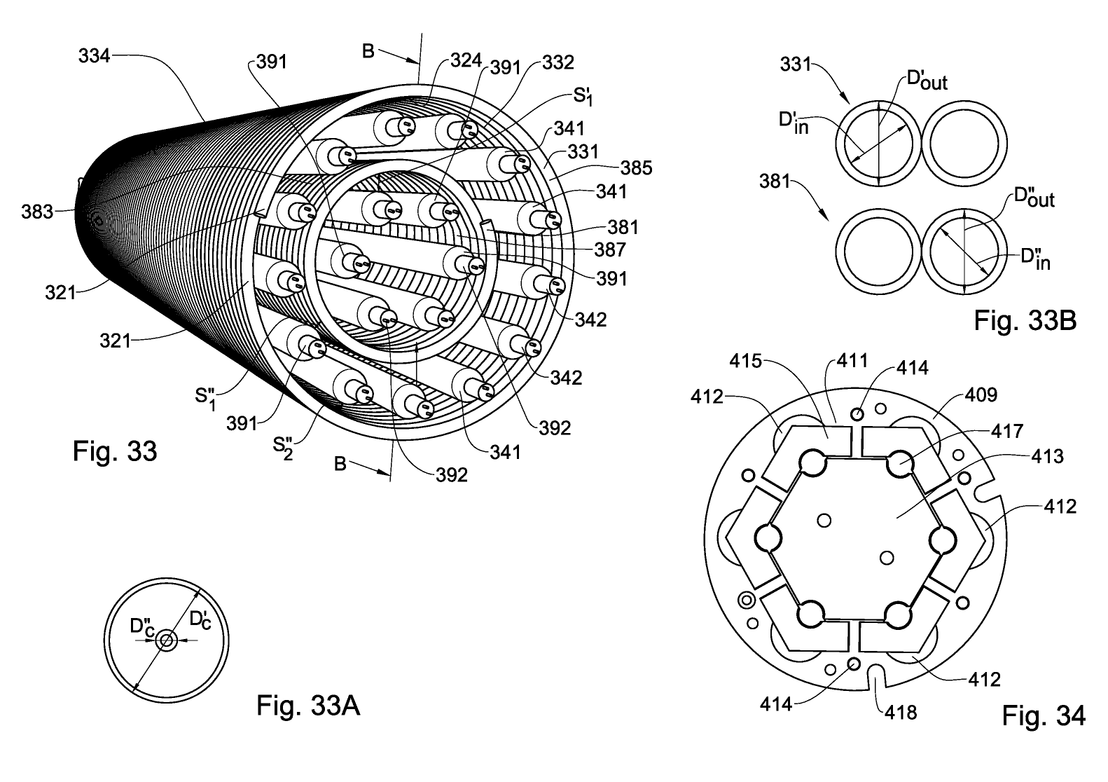

The inlet distributor valve 311 is configured for dividing the incoming liquid between an inner coiled pipe 381 and an outer coiled pipe 331 (both shown in FIG. 33) of the UV treatment system 310, as will be described below in detail. Similarly, the outlet distributor valve 313 is configured for converging the treated liquid from both pipes.

The pre/post-treatment system 302 further comprises a couple of faucets 314 and 315 (FIG. 26) disposed between the outlet flow control faucet 308 and the buffer tank 305, responsible for directing the fluid exiting the UV treatment unit 310 to either repeating the treatment, continuing to post-treatment processes or sewage.

Turning now to FIGS. 26 to 29, there is shown the UV treatment unit 310 and a unit support assembly 316, configured for providing an outward support to the UV treatment unit 310 and for fixing it to the stage 304.

In particular, the unit support assembly 316 comprises three end supports 319, 320 and 350 (FIG. 26), each being fixedly attached to the stage 304 at their bottom portions 319a, 320a and 350a, respectively, and further comprises four horizontal supports 322, 324, 326 and 328, extending substantially parallel to a unit central axis C (FIG. 25) of the UV treatment unit 310, between the end supports 319 and 320, to which they are fixedly attached.

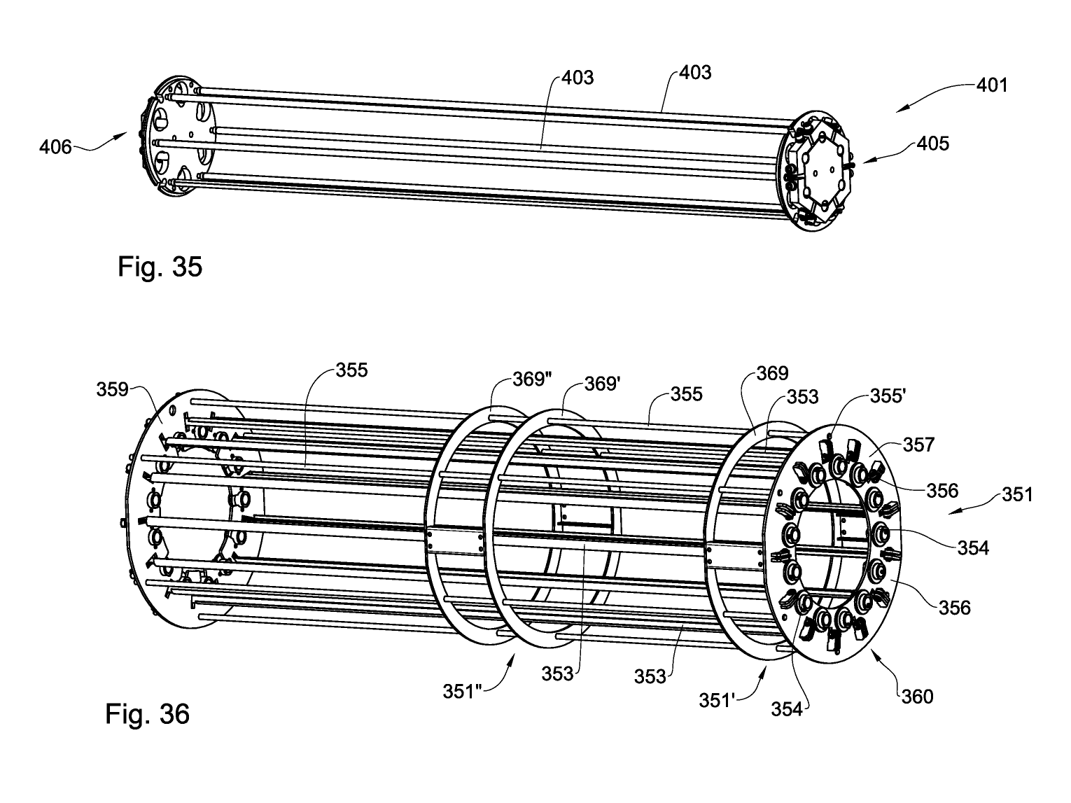

The end support 350 is fixedly attached to a flange assembly 406, referred to below in detail. The UV treatment unit 310 is of an elongated shape and it comprises two main assemblies (best seen in FIGS. 33 to 37):
I. An outer pipe assembly 330, comprising:
   a. an outer coiled pipe 331,
   b. a first group of UV radiation lamps 341, and
   c. an outer supporting assembly 351 (FIG. 36).
II. An inner pipe assembly 380, comprising:
   a. an inner coiled pipe 381,
   b. a second group of UV radiation lamps 391, and
   c. an inner supporting assembly 401 (FIG. 35).

Each of the coiled pipes 330, 380 may be provided with a spinner 21 as described above with reference to FIG. 7D.

The above assemblies extend between the inlet and the outlet 310a and 310b of the UV treatment unit 310 and are disposed concentrically about the unit central axis C, wherein the inner pipe assembly 380 is coaxially inserted within the outer pipe assembly 330. The arrangement being such that an outer coiled pipe 331 surrounds the first radiation lamps 341, which surrounds the inner coiled pipe 381, and which in turn surrounds the second radiation lamps 391 (FIG. 33).

The above arrangement allows the fluids which pass through the outer coiled pipe 331 to be exposed to radiation from the inside by the first radiation lamps 341, and to the fluids which pass through the inner coiled pipe 381 to be exposed to the radiation from the outside by the first radiation lamps 341 and from the inside by the second radiation lamps 391.

The inner pipe 381 and the outer pipe 331 are helically coiled pipes of a kind described above with reference to the unit 10.

The outer coiled pipe 331 has inner and outer diameters D′ᵢₙ and D′ₒᵤₜ, and the wall thickness t′_{w}. The wall thickness t′_{w} of the pipe 331 can be in the range 0.6-1.3, more particularly 0.63-1.27 mm. The inner diameter D′ᵢₙ of the pipe 331 can be in the range of 10-25 mm, and specifically 11 mm, allowing a flow velocity to be in the range of 2-4.5 m/sec.

The coiled pipe 331 has a form of a sleeve with an outer cylindrical surface 334 and an inner cylindrical surface 324 defining a cavity 332, all extending between pipe inlet and outlet 331a and 331b.

The coil length L′ (FIG. 25) can be in the range of 1,200-1,700 mm, and more particularly 1,475 mm.

The number of turns 321 forming the coil of the pipe 331 influences the time of exposure of the fluid to the UV radiation and thus the efficiency of the treatment process, and therefore it is desired to increase it as much as possible, taking into consideration other parameters such as the pipe length, spacing between the turns, pressure drop, organoleptic properties of the product, etc. The number of turns of the coil 331 in the described example of the system can be in the range of 100-130, and specifically 116.

The coil outer diameter D′c can be in the range of 350-400 mm, and specifically 386 mm, and the inner diameter of the pipe D′ᵢₙ and the diameter D′_{C} of the coil can be chosen so as to satisfy Dean ratio, as explained above, i.e. 0.03≦D′ᵢₙ/D′_{C}≦0.1, and specifically 0.03.

The inner coiled pipe 381 has inner and outer diameters D″ᵢₙ and D″ₒᵤₜ, and the wall thickness t″_{w}. The wall thickness t″_{w} of the pipe 381 can be in the range 0.6-1.3, more particularly 0.63-1.27 mm. The inner diameter D″ᵢₙ of the pipe 381 can be in the range of 10-15 mm, and specifically 11 mm, allowing a flow velocity to be in the range of 2-3.5 m/sec.

The inner coiled pipe 381 has a form of a sleeve with an outer cylindrical surface 383 and an inner cylindrical surface 385 defining a cavity 387, all extending between pipe inlet and outlet 331a and 331b.

The coil length L″ and the number of turns 389 are equal to the corresponding values of the outer pipe 331.

The coil outer diameter D″_{c} can be in the range of 180-250 mm, and specifically 228 mm, and the inner diameter of the pipe D″ᵢₙ and the diameter D″_{C} of the coil can be chosen so as to satisfy Dean ratio, as explained above, i.e. 0.03≦D″ᵢₙ/D″_{C}≦0.1, and specifically 0.05.

The lamps 341 and 391 are UV tube-shaped inner lamps, similar to the lamps 51 and 71 described above, and they extend between the inlet and the outlet 310a and 310b of the UV treatment unit 310. The lamps 341 are identical to the lamps 391, or can differ therefrom by some of their properties.

The lamps 341 and 391 have a length in the range of 1,200-1,700 mm, and specifically 1,554 mm (total length) and 1475 (the emitting section) and intensity in the range of 500-1,200 μW/cm².

The lamps 391 of the inner pipe assembly 380 are disposed within the coil cylindrical cavity 387 of the inner pipe 381 and are uniformly distributed around the axis C and spaced from the coil inner cylindrical surface 385 by a distance of s′₁.

The lamps 341 of the outer pipe assembly 330 are disposed within the coil cylindrical cavity 332 of the outer pipe 331 being uniformly distributed around the axis C and spaced from the coil outer cylindrical surface 383 of the inner pipe 381 by a distance s″₁ and from the inner cylindrical surface 324 of the outer pipe 331 by a distance s′₂ (FIG. 33).

In accordance with one example of the presently disclosed subject matter the number of the lamps 341 of the outer pipe assembly 330 can be [in the range of 3-10 and specifically 6 and the number of the lamps 391 of the inner pipe assembly 380 can be in the range of 10-20 and specifically 13].

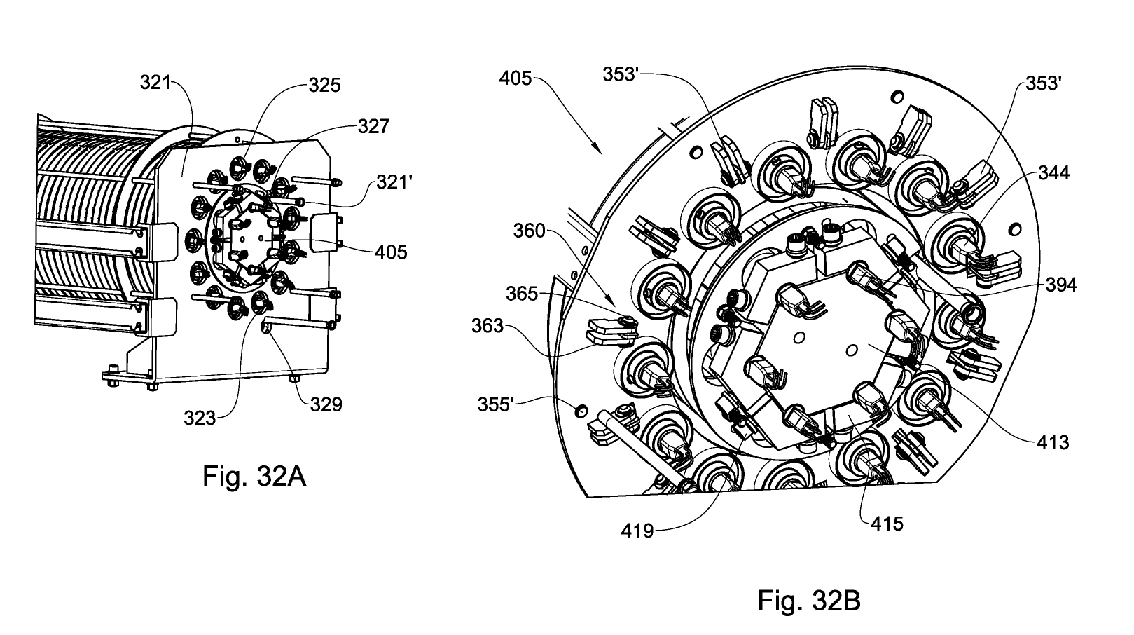

The lamps 341 and 391 are fitted with respective supporting sockets 342 and 392, which are provided with electrical plugs 344 and 394 (FIG. 32B) connecting the lamps to an electrical source (not shown).

As further described in detail, each lamp is mounted separately to the corresponding supporting assembly.

It should be appreciated that the lamps of the inner and outer assemblies 330 and 380 can each have a single radiation source, for example, an elongated cylindrical radiation source having dimensions suitable for being inserted within the corresponding coil cylindrical cavity or an elongated tubular source for surrounding the pipe sub-system.

It should also be appreciated that the inner and outer radiation sub-systems can have identical or different properties, such as light intensity, dimensions, etc.

Referring now to FIG. 36, there is shown an outer supporting assembly 351, configured for holding outer pipe supporting rods 353 and the lamps 341.

The outer supporting assembly 351 is a structure comprising a number of vertically oriented supports for fixedly supporting the outer pipe supporting rods 353 and the lamps 341, and a plurality of horizontally oriented fixation rods 355 connecting the supports, extending coaxially with the assembly central axis C.

In particular, the outer supporting assembly 351 comprises: a first support frame 351′ disposed at the inlet 310a of the UV treatment unit 310 and composed of a first flange 357 and a support ring 361, being fixedly attached thereto, a second flange 359, disposed at the outlet 310b of the UV treatment unit 310, a second support frame 351″ being disposed between the first support frame 351′ and the second flange 359, comprising a couple of support rings 361 and 361″ being fixedly attached to each other, a plurality of fixation rods 355 extending between the first flange 357 and the second flange 359 through the rings 361, 351′ and 361″.

The first flange 357 comprises a plurality of lamp openings 356 for receiving therein the sockets 342 of the lamps 341, which are fixed therein by means of corresponding nuts 354, a plurality of supporting rod openings 359 (not seen), for receiving therein ends 353′ (FIG. 32B) of the outer pipe supporting rods 353, fixed therein by means of a toggle mechanism 360, as detailed below, and a plurality of peripheral openings for fixedly receiving therein ends 355′ of the fixation rods 355.

The second flange 359 (best seen in FIG. 31) comprises a plurality of lamp openings 356′ for receiving therein the sockets 392 of the lamps 391, which are fixed therein by means of corresponding nuts 354′, a plurality of supporting rod openings 359′, for receiving therein ends 353″ of the outer pipe supporting rods 353, fixed therein by means of the toggle mechanism 360, as detailed below, and a plurality of peripheral openings for fixedly receiving therein ends 355″ of the fixation rods 355.

The toggle mechanism 360 comprises a plurality of rib couples 363 (FIG. 32B) for holding therebetween the ends 353′ of the outer pipe supporting rods 353 fixed by means of corresponding fixation screws 365 to the first flange 357, and a plurality of toggle fasteners 367 (FIG. 31), fixed to the second flange 359, and configured to fasten the ends 353″ to the outer pipe supporting rods 353. Such arrangement facilitates the assembly of the outer pipe 331, as further detailed below.

Referring now to FIG. 35, there is shown an inner supporting assembly 401, configured for holding the inner pipe 381 and the lamps 391.

The inner supporting assembly 401 is a structure comprising two flange assemblies 405 and 406 disposed at the inlet and the outlet 310a and 310b respectively, of the UV treatment unit 310, and a plurality of inner pipe supporting rods 403 horizontally oriented and extending coaxially with the assembly central axis C.

The flange assemblies 405 and 406 are configured for fixedly supporting the inner pipe supporting rods 403 and the lamps 391.

The flange assembly 405 (FIG. 34) comprises a holding plate 409 comprising a base portion 411 and an elevating portion 413 extending outwardly therefrom and a plurality of retainers 415 configured to fit the outer shape of the elevating portion 413, and form together therewith openings 417 for receiving therein the sockets 392 of the lamps 391. The retainers 415 are fixed to the elevating portion 413 by means of corresponding fixation screws 419 (FIG. 32B).

The base portion 411 comprises a plurality of lamp openings 412 for the ends 391a of the lamps 391 to pass therethrough and a plurality of rod openings 414 disposed between the lamp openings 412 to receive therein ends 403a of the inner pipe supporting rods 403, which are fixed to the base portion 411.

The base portion 411 further comprises a pipe receiving passage 418 for receiving therein an inlet 381a of the pipe 381.

Similarly, the flange assembly 406 (best seen in FIG. 31) comprises a holding plate 408 comprising a base portion 421 and an elevating portion 423 extending outwardly therefrom and a plurality of retainers 425 configured to fit the outer shape of the elevating portion 423, and form together therewith openings 427 for receiving therein ends of the lamps 391b. The retainers 425 are fixed to the elevating portion 423 by means of corresponding fixation screws 429.

The base portion 421 comprises a plurality of lamp openings 422 for the ends 391b of the lamps 391 to pass therethrough and a plurality of rod openings 424 disposed between the lamp openings 422 to receive therein ends 403b of the inner pipe supporting rods 403, which are fixed to the base portion 421.

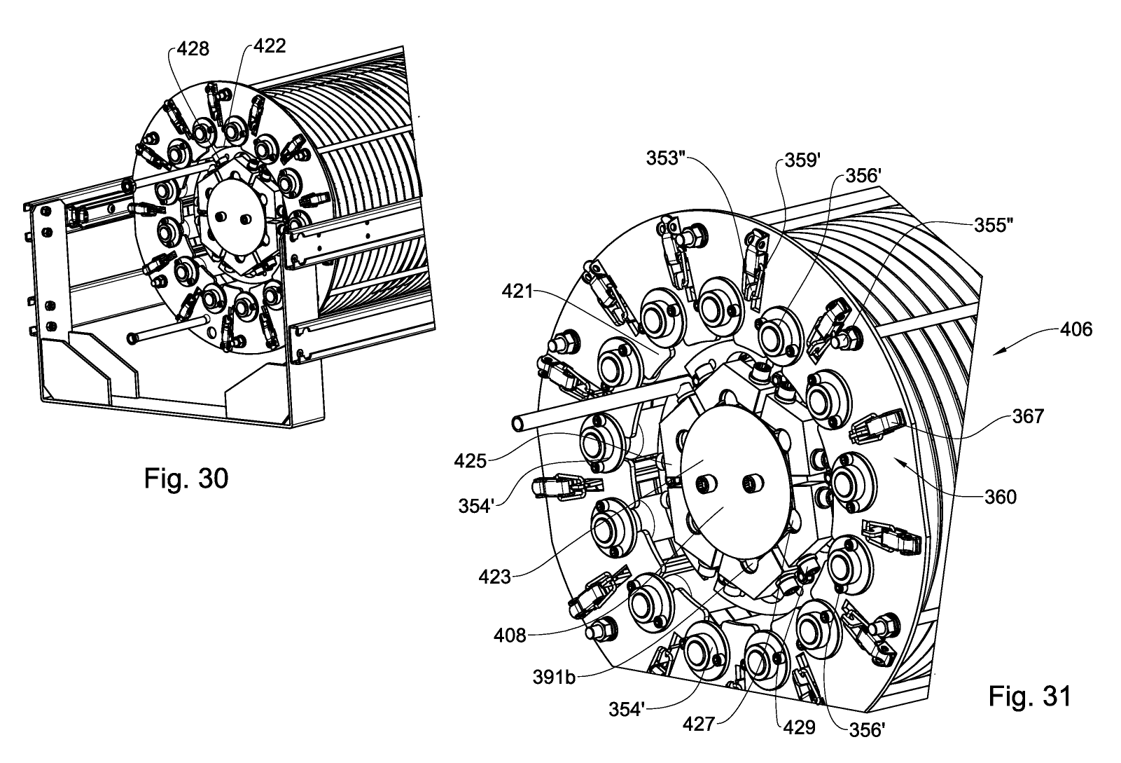

The base portion 421 further comprises a pipe receiving passage 428 (FIG. 30) for receiving therein an outlet 381b of the pipe 381.

The flange assembly 406 can further comprise a cover 427 for covering the elevating portion 423.

Turning back to FIGS. 24, 28 and 29 showing the unit support assembly 316, the end support 319 (best seen in FIG. 32A) comprises a lateral portion 321 having a flange opening 323, a plurality of lamp openings 325 and two pipe openings 327 and 329.

The arrangement is such that the flange assembly 405 is tightly received within the flange opening 323 so that the elevating portion 413 protrudes outwardly from an outer surface 321′ of the lateral portion 321, and the sockets of 342 of the outer lamps 341 extend through the lamp openings 325.

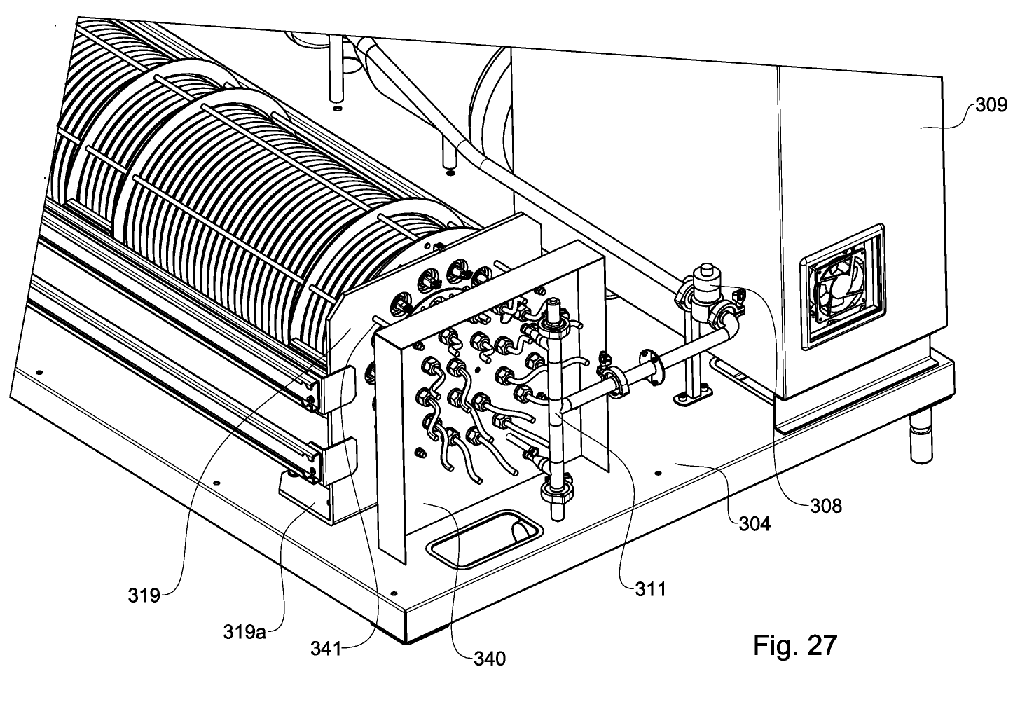

Spaced from lateral portion 321 of the end support 319 there is the separator 340 (FIGS. 24 and 27), which is attached to the lateral portion 321 of the end support 19 by means of separation rods 341.

The arrangement is such that cables of each of the electrical plugs of the lamps 341 and 391 are collected together so as to pass through corresponding openings within the separator 340 so as to extend therefrom to reach the control board 309. Such arrangement allows a direct access to the cables of the electrical plugs and prevents unnecessary movements of the cables.

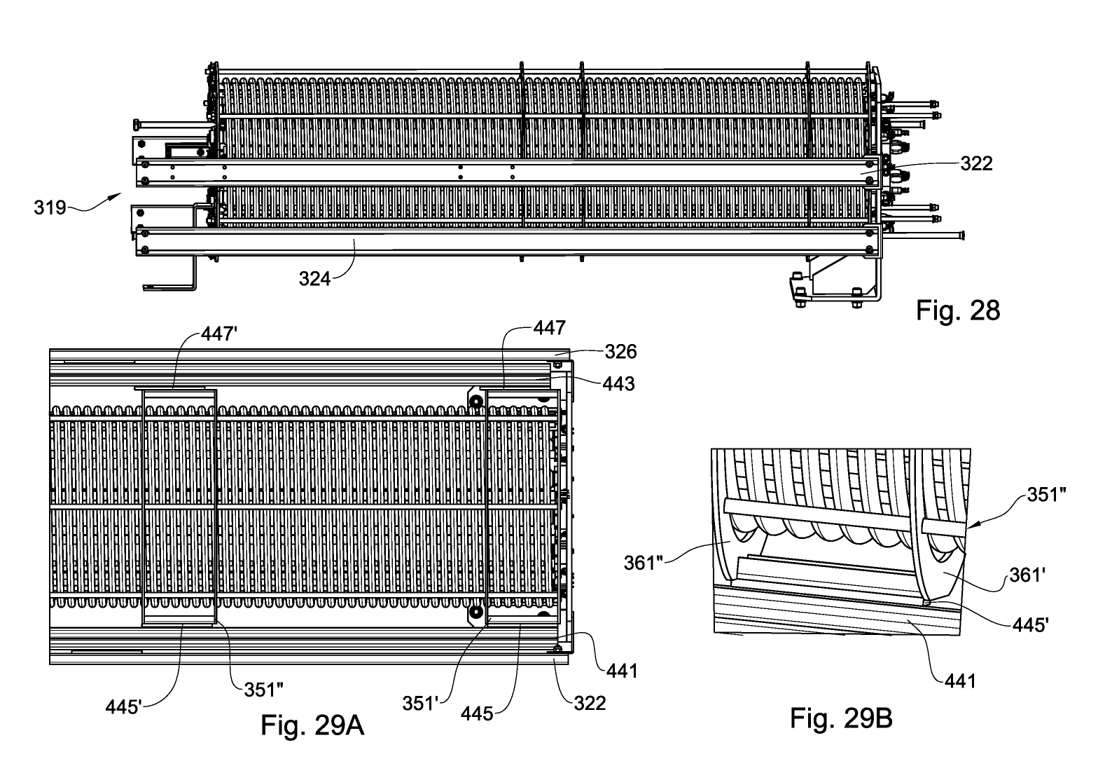

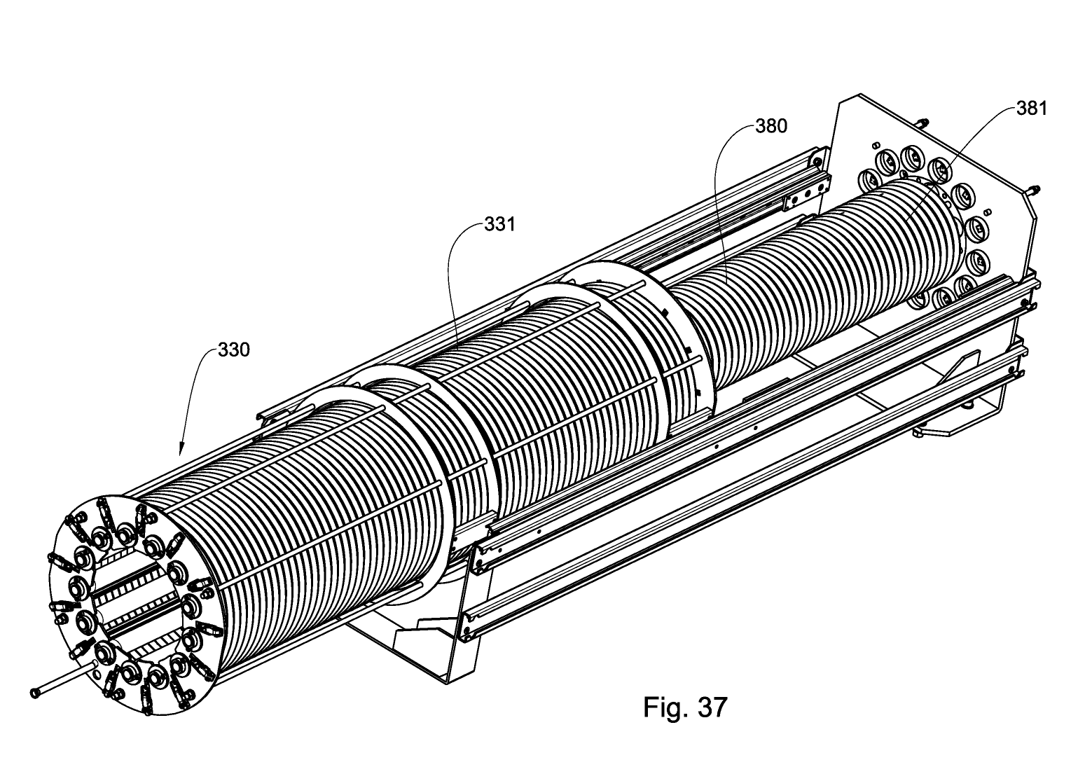

With reference to FIGS. 28, 29A and 29B, the unit support assembly 316 further allows sliding of the outer pipe assembly 330 with respect to the inner pipe assembly 380, as shown in FIG. 37.

In particular, the horizontal supports 322 and 324 are fitted with guide tracks 441 and 443 respectively, and the first support frame 351′ and the second support frame 351″ are fitted with sliding elements 445, 445′, 447 and 447′, respectively, configured for sliding along the guide tracks 441 and 443 along the axis C of the treatment unit 310, exposing thereby the pipe inner assembly 380 (FIG. 37).

The operation of the UV treatment unit 310 is such that a liquid to be treated flows through the inlet distributor valve 311 where it is divided between the inner coiled pipe 381 and the outer coiled pipe 331. The liquid which passes through the outer coiled pipe is treated from the inside by the UV radiation of the lamps 341 and the liquid which passes through the inner coiled pipe 381 is treated from the outside by the UV radiation of the lamps 341 and from the inside by the lamps 391.

The treated liquid then passes through the outlet distributor valve 313 which converges with the treated liquid from both pipes.

In order to make the convergence, the liquid from one pipe has to be treated equally to the liquid from the other pipe. In particular, the absorbance of the treated liquid has to be the same in both pipes, as to satisfy: F₁=F₂, i.e. i₁×t₁=i₂×t₂, wherein F₁ is the UV dose of the liquid of the first pipe and F₂ is the UV dose of the liquid of the second pipe, i is the intensity of the applied UV light from the corresponding lamps and t is the time of the exposure of the corresponding fluid to the above UV light.

The intensity (i.e. the number and the intensity of the lamps) and the exposure time of the outer pipe assembly 330 and the inner pipe assembly 380 have to be such that the above equation is satisfied.

Assuming that the number and the parameters of the lamps and the internal diameters of the pipes D′ᵢₙ and D″ᵢₙ were already determined in accordance with considerations indicated above with respect to the unit 10, the parameter that has to be determined is the time of the exposure.

The time of the exposure depends on the flow rate of the fluid, the internal diameter of the pipe and the distance which the fluid passes within the pipe, determined by the coil outer diameter, i.e. D′_{c} and D″_{c}.

In a specific case that the coil outer diameter D″c of the inner coiled pipe 381 is determined as described with respect to the unit 10, the coil outer diameter D′c of the outer coiled pipe 331 has to be determined accordingly, so as to satisfy the above absorbance equation F₁=F₂.

The system can be fitted with an arrangement for flow rate regulation, i.e. responsible for controlling the flow rate in one or both pipes to assure that the flow rate in both pipes is equal.

It should be appreciated that other designing steps are also possible, as long as the absorbance equation is satisfied.

Finally, the process of assembly of the UV treatment unit 310 is generally summarized below:
a. The inner pipe assembly 381 is assembled by fixing the rods 403 to the flange assembly 405, inserting the inner coiled pipe 391 and mounting the second flange assembly 406; Lamps are being inserted to their place only after all unit 310 is fixated to subassembly 304;
b. The outer supporting assembly 351 by fixing the rods 355 and flanges 369, 369′, 369″ to the flange 357, and fixing the rods 353 to the flange 357;
c. Inserting the outer coiled pipe 331 while the outer pipe supporting rods 353 are loose, fixing the flange 359 and stretching out the outer pipe supporting rods 353 by the mechanism 360;
d. Fixing one end of the inner pipe assembly 380 to the end support 319, assembling the unit support assembly 316 and fixing it to the stage 304;
e. Slidingly inserting the outer pipe assembly 330 along the horizontal supports 322 and 324, fixing to the end support 319;
f. Fixing the flange assembly 406 to the stage by means of the end support 350; and
g. Inserting the lamps 341 and 391.

1. A radiation treatment unit comprising:
a pipe sub-system comprising a helically-coiled pipe for passing therethrough a fluid to be treated and defining therein a cylindrical cavity; and
at least one radiation sub-system configured to generate radiation and disposed so as to expose said pipe sub-system to said radiation;
wherein said pipe is provided with a spinner therewithin, said spinner comprising smoothly twisting spinning surfaces spanning between ends thereof.

2. The radiation treatment unit of claim 1, wherein said spinning surfaces are twisted through substantially 360° between the ends of the spinner.

3. The radiation treatment unit of claim 1, wherein said spinning surfaces span between two helical edges.

4. The radiation treatment unit of claim 3, wherein said helical edges tightly fit within the inner cylindrical surface of pipe.

5. The radiation treatment unit of claim 1, wherein the pitch of the spinning surfaces is substantially constant between the ends of the spinner.

6. The radiation treatment unit of claim 1, wherein the pitch of the spinning surfaces decreases along its length.

7. The radiation treatment unit of claim 1, wherein the spinner is made of stainless steel.

8. The radiation treatment unit according to claim 1, the pipe having a smooth inner surface of a diameter D, the coiled pipe having a coil inner cylindrical surface defining a cylindrical cavity and a coil outer cylindrical surface of a diameter D_{C}, wherein 0.03≦D/D_{C}≦0.1.

9. The radiation treatment unit according to claim 8, wherein the pipe satisfies the condition D/D_{C}=0.05.

10. The radiation treatment unit according to claim 8, wherein D is between about 10 mm and about 35 mm.

11. The radiation treatment unit according to claim 1, said pipe having a linear of between about 1,200 mm and about 1,700 mm.

12. The radiation treatment unit according to claim 1, wherein the pipe has a wall thickness of between about 0.63 mm and about 1.27 mm.

13. The radiation treatment unit according to claim 1, wherein the pipe is made of a Fluoropolymer.

14. The radiation treatment unit according to claim 1, said radiation sub-system comprising at least one radiation source selected from the group including an inner radiation source disposed within the cylindrical cavity and an outer radiation source disposed outside the coil.

15. The radiation treatment unit according to claim 14, wherein said radiation sub-system comprises said inner radiation source and said outer radiation source.

16. The radiation treatment unit according to claim 14, wherein each said radiation source extends along the length of the coil.

17. The radiation treatment unit according to claim 14, wherein each said radiation source comprises one or more ultraviolet lamps.
